(19) United States
(12) Patent Application Publication
(10) Pub. No.: US 2023/0201222 A1
(43) Pub. Date: Jun. 29, 2023

(54) ALLEVIATION OF LIVER INJURY BY ACTIVATING THE SIGNALING PATHWAY MEDIATED BY FARNESOID X RECEPTOR

(71) Applicant: CHILDREN'S HOSPITAL MEDICAL CENTER, Cincinnati, OH (US)

(72) Inventor: Akihiro ASAI, Cincinnati, OH (US)

(73) Assignee: CHILDREN'S HOSPITAL MEDICAL CENTER, Cincinnati, OH (US)

(21) Appl. No.: 17/998,731

(22) PCT Filed: May 12, 2021
(86) PCT No.: PCT/US2021/031940

§ 371 Date: Nov. 14, 2022

Related U.S. Application Data
(60) Provisional application No. 63/129,963, filed on Dec. 23, 2020.
(60) Provisional application No. 63/024,259, filed on May 13, 2020.

(51) Int. Cl. A61K 31/575; A61K 31/133; A61K 31/661; A61K 31/216; A61K 31/42

(52) U.S. Cl. CPC A61K 31/575; A61K 31/133; A61K 31/661; A61K 31/216; A61K 31/42

(57) ABSTRACT

The present disclosure provides methods method for alleviating liver injury using a Farnesoid X receptor (FXR) activator.

### CROSS REFERENCE TO RELATED APPLICATIONS

This application claims the benefit of the filing dates of U.S. Provisional Application No. 63/024,259, filed May 13, 2020 and U.S. Provisional Application No. 63/129,963, filed Dec. 23, 2020, the entire contents of each of which are incorporated by reference herein.

### BACKGROUND OF THE INVENTION

Cholestasis is defined as a decrease in bile flow due to impaired secretion by hepatocytes or to obstruction of bile flow through intra- or extrahepatic bile ducts. Therefore, the clinical definition of cholestasis is any condition in which substances normally excreted into bile are retained. The serum concentrations of conjugated bilirubin and bile salts are the most commonly measured.

Bile acids, the major component of bile, are cholesterol metabolites that are formed in the liver and secreted into the duodenum of the intestine, where they have important roles in the solubilization and absorption of dietary lipids and vitamins. Most bile acids (˜95%) are subsequently reabsorbed in the ileum and returned to the liver via the enterohepatic circulatory system. Hepato-enteric recirculation of bile acids regulates a balance between de novo synthesis and sinusoid-to-canalicular transport of bile acids in hepatocytes. This is mediated by the intracellular accumulation of bile acids. Since bile flow is dependent on efficient bile acid transport by hepatocytes, genetic defects affecting bile acid transporters, which disturb the canalicular export of bile acids and result in cholestasis. The characteristic pattern of clinical presentation includes jaundice, pruritus, elevated serum bile acid levels, fat malabsorption, fat soluble vitamin deficiency, and liver injury.

Cholestasis often does not respond to medical therapy of any sort. Some reports indicate success in children with chronic cholestatic diseases with the use of ursodeoxycholic acid, which acts to increase bile formation and antagonizes the effect of hydrophobic bile acids on biological membranes. Phenobarbital may also be useful in some children with chronic cholestasis.

Treatment of fat malabsorption principally involves dietary substitution. In older patients, a diet that is rich in carbohydrates and proteins can be substituted for a diet containing long-chain triglycerides. In infants, that may not be possible, and substitution of a formula containing medium-chain triglycerides may improve fat absorption and nutrition.

In chronic cholestasis, careful attention must be paid to prevent fat-soluble vitamin deficiencies, which are common complications in pediatric patients with chronic cholestasis. This is accomplished by administering fat-soluble vitamins and monitoring the response to therapy. Oral absorbable, fat-soluble vitamin formulation A, D, E, and K supplementation is safe and potentially effective in pediatric patients with cholestasis.

It is therefore of great interest to develop new models to gain a greater understanding of the pathogenic mechanisms of cholestatic liver diseases, to provide insight into therapeutic targeting in subjects suffering from cholestasis, and to screen for drug candidate for treating the disease.

### SUMMARY OF THE INVENTION

The present disclosure is based unexpected discovery that basolateral transport of exogenous bile acids suppressed the de novo synthesis of endogenous bile acids via the Farnesoid X receptor (FXR) pathway. These findings suggested that FXR activators would be effective in alleviating liver injuries associated abnormal secretion and/or transportation of bile acids.

Accordingly, one aspect of the present disclosure provides a method for alleviating liver injury, comprising administering to a subject in need thereof an effective amount of a Farnesoid X receptor (FXR) activator. Exemplary FXR activators include, but are not limited to, Obeticholic acid (OCA), a 6a-ethyl derivative of the natural human BA chenodeoxycholic acid (CDCA), Chenodeoxycholic acid (CDCA), Obeticholic acid, Fexaramine, and GW 4064. In some embodiments, the subject is a human patient having a cholestatic liver disease. Exemplary cholestatic liver diseases include, but are not limited to, benign recurrent intrahepatic cholestasis type 2, intrahepatic cholestasis of pregnancy, or progressive familial intrahepatic cholestasis type 2. In some embodiments, the subject is a pediatric patient.

Also provided herein are pharmaceutical compositions comprising any of the FXR activators for use in treating a cholestatic liver disease, and uses of such FXR activators for manufacturing a medicament for use in the intended therapy as disclosed herein.

Other features or advantages of the present invention will be apparent from the following drawings and detailed description of several examples, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1A-1D include diagrams showing the generation of BSEP/ABCB11^{R1090X} mutant human iPSCs. FIG. 1A: a diagram of the gene map of BSEP/ABCB11 and location of R1090X, truncating mutation. FIG. 1B: a diagram showing the CRISPR/Cas9 genome editing was designed to replace the codon of CGA (arginine) with TGA (stop codon). FIG. 1C: a gel showing restriction enzyme digestion with BspHI identified correctly targeted clones of iPSCs (SEQ ID NO:1 and SEQ ID NO:2). FIG. 1D: microscopic bright field images of iPSCs. The cloned iPSCs with BSEP-R1090X mutations (BSEP^{R1090X}) showed comparable morphology to the parental iPSC colonies. (Scale bar: 100 μm).

FIGS. 2A-2J include graphs and images showing hepatic differentiation of BSEP^{R1090X} iPSCs and BSEP protein expression. FIG. 2A: a set of graphs showing albumin concentration and albumin secretion. The left panel shows albumin concentration of the culture supernatant in the upper and lower chambers was measured with ELISA. The supernatant was collected 24 hours after medium changes. The right panel shows albumin secretion per i-Hep cell at the final stage of hepatic differentiation. At the final stage of hepatic differentiation, i-Hep were fixed and stained with Hoechst to measure the cellular (nuclear) density by counting nuclei under the fluorescent microscope. All i-Hep exhibited comparable albumin secretion into the culture medium. ns=not significant, *=p<0.05, n=5 or more. FIG. 2B: a graph showing cell density of i-Hep. Hoechst stained nuclei in the captured images were counted by ImageJ tools (n.s., not significant, p>0.05). FIG. 2C: a graph showing CYP3A4 enzymic activity, which was measured with luciferin-PFBE assay (Promega, cat #V8901). i-Hep were incubated with rifampicin (25 μM) for 2 days prior to the experiments. Rifampicin induced CYP3A4 in both Normal and BSEP^{R1090X} i-Hep to the comparable levels. (*: p<0.05, n=3). FIG. 2D: a set of conventional light microscopic images of Hematoxylin and Eosin staining of normal and BSEP^{R1090X} i-Hep. Scale bar: 50 uM. FIG. 2E: a set of images showing immunofluorescent staining of normal and BSEP^{R1090X} i-Hep at the final stage of the differentiation protocol. Hepatocyte markers, HNF4a and CPS1, were detected both in normal and BSEP^{R1090X}. An endoderm marker of E-cadherin was detected on cell membrane. A tight junction protein, ZO1, was located at borders of cells. Nuclei were stained with Hoechst. (Scale bar: 10 μm) FIG. 2F: a set of images showing immunofluorescent staining of HNF4a, CSP1, E-cadherin, ZO-1 in BSEP^{patient} i-Hep. (scale bar: 10 μm). FIG. 2G: a graph showing gene expression of hepatic differentiation markers in i-Hep. Marker genes of hepatocyte differentiation were compared by quantitative PCR after normalized to 18S rRNA. At the final stage of differentiation, total RNA was extracted from i-Hep. RNA extracts from primary cultured human hepatocytes were used as control and reference for relative expression of hepatic genes (no marks: n.s., *=p<0.05, n=3 or more, the comparison to primary hepatocytes was not displayed). 18S rRNA housekeeping gene expression did not differ between cell types (p>0.05). FIG. 2H: a set of images of a western blotting to detect proteins of normal BSEP and truncated BSEP^{R1090X} from cell lysates of i-Hep. BSEP^{R1090X} i-Hep showed a faint band at the lower level compared to the normal i-Hep lysate. Na-K ATPase (ATP1A1) was included as a loading control. FIG. 2I: a set of images of a western blotting to detect proteins of BSEP from cell lysates of i-Hep by using the antibody detecting C-terminus of BSEP. BSEP^{R1090X} and BSEP^{patient} i-Hep showed no band compared to the normal i-Hep lysate. Na-K ATPase (ATP1A1) was included as a loading control. FIG. 2J: an immunofluorescent image of liver tissue in paraffin sections from a healthy subject and the patient with BSEP^{R1090X} truncating mutation. BSEP is localized at the canalicular membrane structure in the hepatocytes of a healthy subject. The protein with BSEP^{R1090X} mutation is localized in the cytosol, with a clustering pattern, in the hepatocytes of the patient with PFIC2. (Scale bar: 10 μm)

FIGS. 3A-3C include electron microscopic images showing the cellular ultrastructure of BSEP^{R1090X} i-Hep recapitulates the abnormalities observed in the liver tissue of the patient with PFIC2. FIG. 3A: a set of electron microscopic images of normal (left column) and BSEP^{R1090X} i-Hep (right column). Cells on the Transwell membrane were cross-sectioned. Normal i-Hep showed dense microvilli on the apical surface whereas BSEP^{R1090X} i-Hep showed sparse microvilli (black arrows). Basolateral membrane irregularity with wider interstitial space between hepatocytes was observed in BSEP^{R1090X} (white arrowheads). FIG. 3B: a set of graphs showing morphometric analysis of microvilli in the EM images. The density of apical microvilli was counted per cell (left). The length of microvilli was measured by morphometric tools in ImageJ software and averaged per cell (right). (*=p<0.05, 30 cells per i-Hep). FIG. 3C: a set of electron microscopic images of liver tissues from a healthy subject (left column) and the patient with PFIC2 (right column). The hepatocytes of the patient's liver showed decreased microvilli in the bile canaliculus (arrows) and wider interstitial space between basolateral membranes of adjacent cells (arrowheads). (Scale bar: 2 μm).

FIGS. 4A-4I include graphs and images showing the basolateral-to-apical transport of TCA in BSEP^{R1090X} i-Hep. FIG. 4A: a diagram showing the experimental schemes of exogenous TCA transport from the lower chamber to the upper chamber. FIG. 4B: a graph showing the amount of bile acid in the upper chamber was measured at 24 h and 48 h after loading TCA in the lower chamber. (*p<0.05, n=5). FIG. 4C: a graph showing the percentage fraction of the sum of bile acids measured from the upper and lower chamber in a well at 0, 24, 48 hours after loading of TCA. Grey: Percentage fraction of bile acids measured in the lower chamber. Black: in the upper chamber. FIG. 4D: a set of graphs showing the amount of bile acid in the upper chamber and percentage fraction of the sum of bile acids. The amount of bile acid in the upper chamber was measured at 24 h and 48 h after loading TCA in the lower chamber. (*p<0.05, n=5). The percentage fraction of the sum of bile acids measured from the upper and lower chamber in a well at 0, 24, 48 hours after loading of TCA. The left panel shows change of bile acids in the upper chamber over time. The right panel shows the bile acid levels in the upper and lower chambers over time. FIG. 4E: a diagram showing the experimental schemes of TCA transport from the upper chamber to the lower chamber. FIG. 4F: a graph showing the mass of bile acid in culture medium in the lower chamber, 24 h and 48 h after loading TCA in the upper chamber. (*p<0.05, n=5). FIG. 4G: a graph showing the percentage fraction of measured bile acid in a well at 0, 24, 48 hours after loading of TCA. Grey: Percentage fraction of bile acids measured in the lower chamber. Black: in the upper chamber. FIG. 4H: a graph showing the monolayer barrier function measured with trans-epithelial electrical resistance (TEER) between upper and lower chamber via monolayer and transwell membrane. (ns: not significant, *: p<0.05, n=5 or more). FIG. 4I: a graph showing that cell viability was comparable between normal and BSEP^{R1090X} i-Hep at the end of assays of FIGS. 4A and 4E. (p>0.05, n=3 or more).

FIGS. 5A-5D include diagrams and graphs showing the intrahepatic accumulation of D4-TCA in BSEP^{R1090X} i-Hep during transcellular transport. FIG. 5A: a diagram and graph showing the transport assay of isotope labelled TCA (D4-TCA) to determine intracellular accumulation of TCA over a 24 hour-period. D4-TCA (1 μM) was added into the lower chamber. The amount of TCA was quantified by mass spectrometry in the cell lysates collected at 4, 12, and 24 hours after loading. The amount of D4-TCA is calculated per well. (* p<0.05, n=4). FIG. 5B: a diagram and graph showing the uptake assay of D4-TCA. D4-TCA (10 μM) was added into the lower chamber and cell lysates were collected after 5 min and 15 min incubation with or without sodium in the culture medium. Without sodium in culture medium, D4-TCA was not taken up by the i-Hep. (*: p<0.05, n=3). FIG. 5C: a graph showing that cell viability was comparable between normal and BSEP^{R1090X} i-Hep at the end of assays of FIG. 5A. (p>0.05, n=3 or more). FIG. 5D: a graph showing that cell viability was comparable between normal and BSEP^{R1090X} i-Hep, with and without Na+ at the end of assays for FIG. 5B. (p>0.05, n=3 or more).

FIGS. 6A-6I include a diagram and graphs showing BSEP^{R1090X} i-Hep exports intracellular TCA back into the lower chambers via basolateral MRP4. FIG. 6A: a diagram and graphs showing the wash-out assay to determine the transport (efflux) direction of intracellular D4-TCA. After 1 hour of D4-TCA incubation in the lower chamber (10 μM), i-Hep cells were washed with medium and placed in a fresh medium. The intracellular D4-TCA was exported into the fresh medium in the upper and lower chambers and measured at 5, 15, 30 and 60 minutes by mass spectrometry. BSEP^{R1090X} i-Hep showed basolateral excretion of TCA as opposed to normal i-Hep which excretes TCA apically. (*: p<0.05, n=5 or more). FIG. 6B: a graph showing the gene expressions of hepatic ABC transporters in i-Hep cells at the final stage of differentiation were measured by quantitative real-time PCR (n=4). After normalized to 18S rRNA, each gene expression level was shown relative to the expression level in normal i-Hep. When compared to normal i-Hep (*p<0.05), the BSEP^{R1090X} i-Hep expressed more ABCC4/MRP4. 18S rRNA housekeeping gene expression did not differ between cell types (p>0.05). FIG. 6C: a graph showing the wash-out assay to determine the role of MRP4 in intracellular-to-basolateral export of D4-TCA by using MRP4 inhibitor (Ceefourin1). After 1 hour of D4-TCA incubation in the lower chamber (10 μM), i-Hep cells were washed and placed in a fresh medium with or without MRP4 inhibitor. The exported D4-TCA in the lower chamber was measured by mass spectrometry at 5, 15, and 30 minutes. At 15 and 30 min, MRP4 inhibitor decreased D4-TCA export towards lower chamber (* p<0.05, n=4 or more). FIG. 6D: a graph showing that cell viability was comparable between normal and BSEP^{R1090X} i-Hep at the end of assays of FIG. 6A. (p>0.05, n=3 or more). FIG. 6E: a graph showing that cell viability was comparable between normal and BSEP^{R1090X}i-Hep, with and without MRP4 inhibitor (Ceefourin) at the end of assays for FIG. 6C. (p>0.05, n=3 or more). FIG. 6F: a set of images showing liver paraffin sections that were co-stained with anti-MRP4 and anti-β-catenin antibodies and visualized with immunofluorescent secondary antibodies. MRP4/ABCC4 was detected on the plasma membrane of hepatocytes from the patient with PFIC2 and colocalized with β-catenin (arrows). MRP4 was not detected in hepatocytes from the healthy subject, but β-catenin was detected on the plasma membrane (arrowheads). (scale bar: 1p m) FIG. 6G: a graph showing gene expressions of SLC transporters in i-Hep cells at the final stage of differentiation that were measured by quantitative real-time PCR (n=4). After normalized to 18S rRNA, each gene expression level was shown relative to the expression level in normal i-Hep. When compared to normal i-Hep (*p<0.05), the BSEP^{R1090X} i-Hep expressed more SLC51B and SLCO3A1 and less SLC51A. 18S rRNA housekeeping gene expression did not differ between cell types (p>0.05). FIG. 6H: a graph showing a wash out assay that was used to determine the role of OST in intracellular-to-basolateral export of D4-TCA by using an inhibitor of OST (clofazimine, 30 μM). After 1 hour of D4-TCA incubation in the lower chamber (10 μM), i-Hep cells were washed and placed in a fresh medium with or without an OST inhibitor. The exported D4-TCA in the lower chamber was measured by mass spectrometry at 5, 15, and 30 minutes and displayed. At 15 and 30 min, the OST inhibitor decreased D4-TCA export towards the lower chamber (* p<0.05, n=4 or more). FIG. 6I: a graph showing a wash out assay that was used to determine the role of OST in intracellular-tobasolateral export of D4-TCA by using an inhibitor of OATP3a1 (prostaglandin E2, 10 μM) (Adachi et al., 2003). After 1 hour of D4-TCA incubation in the lower chamber (10 μM), i-Hep cells were washed and placed in a fresh medium with or without an OATP3a1 inhibitor. The exported D4-TCA in the lower chamber was measured by mass spectrometry at 5, 15, and 30 minutes and displayed. OATP3a1 inhibitor did not alter D4-TCA export towards the lower chamber in BSEP^{R1090X} i-Hep (p>0.05, n=5).

FIGS. 7A-7L include diagrams and graphs showing that maturing BSEP^{R1090X} i-Hep adapt export synthesized bile acids via the basolateral membrane and respond to exogenous bile acids FIG. 7A: a graph showing the gene expression of CYP7a in i-Hep that was measured by RT-PCR at the last stages of differentiation. In both normal and BSEPR^{R1090X} i-Hep, CYP7a expression increased from Day 17 of culture to Day 21. The fold change of gene expression was based to the values of day 17. (*p<0.05: Day 17 vs Day 21, n=3) FIG. 7B: a graph showing the amount of endogenous taurocholic acid (TCA) exported into the upper chamber (black) and lower chamber (grey) that was measured by mass spectrometry. After the incubation in fresh culture medium for 48 hours, the TCA concentration in the culture supernatant from the upper and lower chambers was determined. Normal i-Hep exported endogenous TCA towards the upper chamber (apical domain) whereas BSEP^{R1090X} i-Hep towards the lower chamber (basolateral domain). Total amount of TCA synthesized by BSEP^{R1090X} i-Hep was less than normal i-Hep. (*=p<0.05) FIG. 7C: a graph showing the amount of intracellular TCA that was measured from cell lysates after 48 hours incubation. Intracellular TCA in normal and BSEP^{R1090X} i-Hep were comparable. FIG. 7D: a graph showing that Sitaxentan (BSEP inhibitor) inhibited the apical export of endogenous bile acids on normal i-Hep. The amount of endogenous taurocholic acid (TCA) exported into the upper chamber and lower chamber was measured by mass spectrometry. After incubation in a fresh culture medium with or without sitaxentan (100 μM) for 48 hours, the TCA concentration in the culture supernatant from the upper and lower chambers was determined. Normal i-Hep without sitaxentan exported endogenous TCA towards the upper chamber (apical domain), whereas i-Hep with sitaxentan exported less endogenous TCA towards the upper chamber. The amount of TCA exported towards the lower chamber were comparable between i-Hep with and without sitaxentan. (*=p<0.05). FIG. 7E: an image showing a schematic description of experiments design in normal i-Hep. Labelled TCA, D4-TCA, was added to the lower chamber. After the incubation, TCA (endogenous and D4-TCA) in the culture medium was measured separately. FIG. 7F: a graph showing the amount of endogenous TCA secreted into the upper and lower chambers that was measured in the conditions cultured with or without exogenous D4-TCA. The exogenous D4-TCA suppressed endogenous synthesis of TCA. (*=p<0.05) FIG. 7G: an image showing a schematic description of experiments design in BSEP^{R1090X} i-Hep. FIG. 7H: a graph showing the amount of endogenous TCA secreted into the upper and lower chambers that was measured in the conditions cultured with or without exogenous D4-TCA. FIG. 7I: a graph showing the intracellular TCA, endogenous and D4-TCA that was measured separately from the cell lysate after the incubation. Exogenous D4-TCA accumulated in normal and BSEP^{R1090X} i-Hep is comparable. (ns: p>0.05). FIG. 7J: a set of graphs that show the gene expression of the FXR pathway was determined by RT-PCR. In both normal and BSEP^{R1090X} i-Hep, CYP7a was down-regulated and SHP was up-regulated when D4-TCA was added into the lower chamber for 12 h and 24 h. No significant change was found in FXR expression. The fold change of gene expression was based to the values in the condition cultured without D4-TCA. (*p<0.05, n=3 or more). FIG. 7K: The amount of endogenous TCA secreted into the upper and lower chambers was measured in the conditions with or without FXR agonist, obeticholic acid (OCA, 10 μM), in normal and BSEP^{R1090X} i-Hep. Obeticholic acid suppressed the endogenous synthesis of TCA. (*=p<0.05, n=4, black: lower chambers cultured with vs. without OCA, blue: upper chambers cultured with vs. without OCA, purple: upper chamber vs. lower chamber of each i-Hep). FIG. 7L: Intracellular TCA, endogenous and D4-TCA, measured separately from the cell lysate after the incubation with vs. without OCA. OAC suppressed intracellular TCA in normal and BSEP^{R1090X} i-Hep. (*=p<0.05, n=4)

FIGS. 8A-8B include a model representing mechanism regulating de novo bile acid synthesis in BSEP deficient hepatocytes FIG. 8A: a diagram showing in normal hepatocytes, synthesized bile acids are exported to the bile canaliculus and return to the sinusoid by the hepato-enteric circulation (1). The bile acids in the sinusoid are taken up by hepatocytes and suppress de novo synthesis mediated by the intracellular concentration of bile acids (2 and 3). FIG. 8B: a diagram showing in BSEP deficient hepatocytes, synthesized bile acids are exported to the sinusoid and accumulate in the systemic circulation (1). When taken up from the sinusoid, the intracellular bile acids suppress de novo bile acid synthesis while being exported to the sinusoid via the basolateral membrane (2 and 3).

FIGS. 9A-9E include showing generation of BSEP/ABCB11^{R1090X} mutant human iPSCs from another iPSC clone (clone code: TkDA3) and hepatic differentiation of the BSEP^{R1090X}-iPSC, as well as dynamics of the bile acid transport in BSEP^{R1090X} i-Hep (TkDA3). FIG. 9A: a graph showing that hepatic differentiation of the BSEP^{R1090X}-(TkDA3) iPSC was comparable to normal (TkDA3)iPSC. Albumin secretion per i-Hep cell during the last 8 days of hepatic differentiation was compared. Normal and BSEP^{R1090X} i-Hep exhibited comparable albumin secretion into the culture medium. FIG. 9B: a diagram and graph showing experimental schemes of exogenous TCA transport from the lower chamber to the upper chamber. The amount of bile acid in the medium of the upper chamber was measured by mass spectrometry at 4 h, 12 h, and 24 h after loading isotope labelled TCA (D4-TCA) in the lower chamber. (*p<0.05, n=5). FIG. 9C: a diagram and graph showing that during the above transport assay of D4-TCA, the intracellular accumulation of TCA over a 24 hour period was determined. The amount of TCA was quantified by mass spectrometry in the cell lysates collected at 4, 12, and 24 hours after loading. The amount of D4-TCA is calculated per well. (* p<0.05, n=5). FIG. 9D: a diagram and graph of an uptake assay of D4-TCA. D4-TCA (10 μM) was added into the lower chamber and cell lysates were collected after 5 min and 15 min incubation with or without sodium in the culture medium. FIG. 9E: a diagram and graphs showing a wash-out assay used to determine the transport (efflux) direction of intracellular D4-TCA in normal vs. BSEP^{R1090X} i-Hep (TkDA3). After 1 hour of D4-TCA incubation in the lower chamber (10 μM), i-Hep cells were washed with medium and placed in a fresh medium. The intracellular D4-TCA was exported into the fresh medium in the upper and lower chambers and measured at 0.5, 1, 2, and 4 hours by mass spectrometry. BSEP^{R1090X} i-Hep showed basolateral excretion of D4-TCA as opposed to the apical excretion seen in normal i-Hep. (*: p<0.05, n=4 or more).

FIGS. 10A-10C include graphs showing de novo bile acid synthesis of BSEP^{R1090X} i-Hep (TkDA3) and the response of FXR related genes to exogenous bile acids. FIG. 10A: a graph showing the amount of endogenous taurocholic acid (TCA) exported into the upper chamber and lower chamber that was measured by mass spectrometry. After incubation in a fresh culture medium for 48 hours, the TCA concentration in the culture supernatant from the upper and lower chambers was determined. FIG. 10B: a graph showing the amount of intracellular TCA that was measured from cell lysates after 48 hours of incubation. Intracellular TCA in normal (TkDA3) was more than that in BSEP^{R1090X} i-Hep (TkDA3). (*=p<0.05, n=3 or more). FIG. 10C: a set of graphs showing gene expression of the FXR pathway that was determined by RT-PCR.

### DETAILED DESCRIPTION OF THE INVENTION

Genetic defects affecting bile acid transport pathways present in several clinical phenotypes including Progressive Familial Intrahepatic Cholestasi (PFIC), Benign Recurrent Intrahepatic Cholestasis (BRIC), and Intrahepatic Cholestasis of Pregnancy (ICP). Progressive familial intrahepatic cholestasis (PFIC) is a class of chronic cholestasis disorders that begin in infancy and usually progress to cirrhosis within the first decade of life. The average age at onset is 3 months, although some patients do not develop jaundice until later, even as late as adolescence. PFIC can progress rapidly and cause cirrhosis during infancy or may progress relatively slowly with minimal scarring well into adolescence. Few patients have survived into the third decade of life without treatment.

PFIC types 1 and 2 are rare, but the exact frequency is unknown. Incidence is estimated at 1:50,000 to 1:100,000 births. All forms of progressive familial intrahepatic cholestasis are lethal in childhood unless treated. Morbidity is the result of chronic cholestasis. Pruritus is more pronounced in PFIC types 1 and 2 and often occurs out of proportion to the level of jaundice, which is often low grade and can wax and wane. The pruritus may be disabling and usually does not respond to medical therapy. Greater understanding of individualized pathways driving disease-causing pathologies and response to therapy, and the clinical translation of these data, is needed to design personalized management strategies at an early stage of the disease.

The present disclosure is based, at least in part, that BSEP deficient hepatocytes can achieve homeostasis of bile acids concentration of the systemic circulation by down-regulating de novo bile acid synthesis via the uptake and export of bile acids on the basolateral domain, while preventing accumulation of intracellular bile acid. Further, it is reported herein that the Farnesoid X receptor (FXR) pathway may be involved in the de novo synthesis of endogenous bile acids, suggesting that FXR activators could benefit treatment of liver diseases, for example, cholestatic liver diseases.

Accordingly, provided herein are methods for alleviating liver injury in a subject in need thereof using one or more FXR activators.

### FXR Pathway Activators and Pharmaceutical Compositions Comprising Such

Farnesoid X receptor (FXR), also known as nuclear receptor subfamily 1, group H, member 4 (NR1H4), is a nuclear receptor that is encoded by the NR1H4 gene in humans. FXR is expressed at a high level in the liver and intestine. Chenodeoxycholic acid and other bile acids are ligands for FXR. Upon binding to the bile acid ligand, activated FXR translocates to the cell nucleus, forms a dimer, and binds hormone response elements on DNAs to modulate gene expression, for example, suppressing the expression of cholesterol 7 alpha-hydroxylase (CYP7A1) and/or up-regulating the expression and activity of epithelial transport proteins involved in fluid homeostasis in the intestine such as the cystic fibrosis transmembrane conductance regulator (CFTR).

As used herein, an FXR pathway activator refers to a molecule (e.g., a small molecule, a peptide or polypeptide, a nucleic acid, or a lipid) that activates the FXR signaling pathway. Non-limiting examples of suitable FXR activators include ethanolamine, phosphoethanolamine, phosphatidylethanolamine, obeticholic acid (OCA), a 6α-ethyl derivative of the natural human BA chenodeoxycholic acid (CDCA), Chenodeoxycholic acid (CDCA), Fexaramine, and GW 4064.

Any of the FXR pathway activators as disclosed herein may be mixed with one or more pharmaceutically acceptable excipients for form a pharmaceutical composition, which can be used in the treatment methods disclosed herein. “Acceptable” means that the carrier must be compatible with the active ingredient of the composition (and preferably, capable of stabilizing the active ingredient) and not deleterious to the subject to be treated. Suitable carriers include microcrystalline cellulose, mannitol, glucose, defatted milk powder, polyvinylpyrrolidone, and starch, or a combination thereof.

To facilitate delivery, any of the FXR pathway activator can be conjugated with a chaperon agent. As used herein, “conjugated” means two entities are associated, preferably with sufficient affinity that the therapeutic benefit of the association between the two entities is realized. Conjugated includes covalent or noncovalent bonding as well as other forms of association, such as entrapment of one entity on or within the other, or of either or both entities on or within a third entity (e.g., a micelle).

The chaperon agent can be a naturally occurring substance, such as a protein (e.g., human serum albumin, low-density lipoprotein, or globulin), carbohydrate (e.g., a dextran, pullulan, chitin, chitosan, inulin, cyclodextrin or hyaluronic acid), or lipid. It can also be a recombinant or synthetic molecule, such as a synthetic polymer, e.g., a synthetic polyamino acid. Examples of polyamino acids include polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl) methacrylamide copolymer (HMPA), polyethylene glycol (PEG), polyvinyl alcohol (PVA), polyurethane, poly(2-ethylacryllic acid), N-isopropylacrylamide polymers, and polyphosphazine.

In one example, the chaperon agent is a micelle, liposome, nanoparticle, or microsphere, in which the FXR pathway activator is encapsulated. Methods for preparing such a micelle, liposome, nanoparticle, or microsphere are well known in the art. See, e.g., U.S. Pat. Nos. 5,108,921; 5,354,844; 5,416,016; and 5,527,5285.

In another example, the chaperon agent serves as a substrate for attachment of one or more of a fusogenic or condensing agent. A fusogenic agent is responsive to the local pH. For instance, upon encountering the pH within an endosome, it can cause a physical change in its immediate environment, e.g., a change in osmotic properties, which disrupts or increases the permeability of the endosome membrane, thereby facilitating release of the antisense oligonucleotide into host cell's cytoplasm. A preferred fusogenic agent changes charge, e.g., becomes protonated at a pH lower than a physiological range (e.g., at pH 4.5-6.5). Fusogenic agents can be molecules containing an amino group capable of undergoing a change of charge (e.g., protonation) when exposed to a specific pH range. Such fusogenic agents include polymers having polyamino chains (e.g., polyethyleneimine) and membrane disruptive agents (e.g., mellittin). Other examples include polyhistidine, polyimidazole, polypyridine, polypropyleneimine, and a polyacetal substance (e.g., a cationic polyacetal).

A condensing agent interacts with the antisense oligonucleotide, causing it to condense (e.g., reduce the size of the oligonucleotide), thus protecting it against degradation. Preferably, the condensing agent includes a moiety (e.g., a charged moiety) that interacts with the oligonucleotide via, e.g., ionic interactions. Examples of condensing agents include polylysine, spermine, spermidine, polyamine or quarternary salt thereof, pseudopeptide-polyamine, peptidomimetic polyamine, dendrimer polyamine, arginine, amidine, protamine, cationic lipid, cationic porphyrin, and alpha helical peptide. A pharmaceutical composition comprising a FXR activating agent can be formulated according to routes of administration, including, e.g., parenteral administration, oral administration, buccal administration, sublingual administration, and topical administration.

In some embodiments, the pharmaceutical composition or formulation is suitable for oral, buccal or sublingual administration, such as in the form of powder, tablets, capsules, ovules, elixirs, solutions or suspensions, which may contain flavoring or coloring agents, for immediate-, delayed- or controlled-release applications.

Suitable tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the FXR activating agent may be combined with various sweetening or flavoring agents, coloring matter or dyes, with emulsifying and/or suspending agents and with diluents such as water, ethanol, propylene glycol and glycerin, and combinations thereof. For powder, the FXR activating agent may be combined with various sweetening or flavoring agents, coloring matter or dyes, with emulsifying and/or suspending agents and with diluents such as water, ethanol, propylene glycol and glycerin, and combinations thereof.

In some embodiments, the pharmaceutical compositions or formulations are for parenteral administration, such as intravenous, intra-arterial, intra-muscular, subcutaneous, or intraperitoneal administration.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from 3 to 9). The preparation of suitable parenteral formulations under sterile conditions is readily accomplished by standard pharmaceutical techniques well-known to those skilled in the art.

The formulations of any aspects described herein may be presented in unit-dose or multi-dose containers, for example sealed ampoules or vials, and may be stored in a freeze-dried (lyophilized) condition requiring only the addition of the sterile liquid carrier immediately prior to use.

Any of the pharmaceutical compositions may be formulated as modified release dosage forms, including delayed-, extended-, prolonged-, sustained-, pulsed-, controlled-, accelerated- and fast-, targeted-, programmed-release, and gastric retention dosage forms. These dosage forms can be prepared according to conventional methods and techniques known to those skilled in the art.

### Alleviation of Liver Injury

To perform any of the methods disclosed herein, an effective amount of one or more of the FXR pathway activator or a pharmaceutical composition comprising such may be administered to a subject in need of the treatment.

The term “treating” as used herein refers to the application or administration of a composition including one or more active agents to a subject, who has liver injury, a symptom of liver injury, or a predisposition toward liver injury, with the purpose to cure, heal, alleviate, relieve, alter, remedy, ameliorate, improve, or affect the disease, the symptoms of the disease, or the predisposition toward the disease.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

An “effective amount” is that amount of an FXR activator agent that alone, or together with further doses, produces the desired response, e.g. eliminate or alleviate symptoms, prevent or reduce the risk of flare-ups (maintain long-term remission), and/or restore quality of life. The desired response is to inhibit the progression of the disease. This may involve only slowing the progression of the disease temporarily, although more preferably, it involves halting the progression of the disease permanently. This can be monitored by routine methods or can be monitored according to diagnostic and prognostic methods discussed herein. The desired response to treatment of the disease or condition also can be delaying the onset or even preventing the onset of the disease or condition.

Such amounts will depend, of course, on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an FXR pathway activator or a pharmaceutical composition comprising such is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Any of the methods described herein can further comprise adjusting the liver injury treatment performed to the subject based on the results obtained from the methods disclosed herein (e.g., based on gene signatures disclosed herein). Adjusting treatment includes, but are not limited to, changing the dose and/or administration of the FXR activating agent used in the current treatment, switching the current medication to a different FXR activating agent, or applying a new liver injury therapy to the subject, which can be either in combination with the current therapy or replacing the current therapy.

A subject according to any of the methods described herein can be a mammal, e.g., a human patient having, suspected of having, or at risk a liver injury. A subject having liver injury may be diagnosed based on clinically available tests and/or an assessment of the pattern of symptoms in a subject and response to therapy. In some instances, the subject may exhibits a genetic mutation in a gene responsible for contributing to liver injury (e.g., ABCB11/BSEP). In some embodiments, the subject has or is suspected of having progressive familial intrahepatic cholestasis type 2. In some embodiments, the subject is a pediatric subject. A pediatric subject may be 10 of 18 years old or below. In some examples, a pediatric patient may have an age range of 0-12 years, e.g., 6 months to 8 years old or 1-6 years.

Dosage and dosing schedule of the FXR pathway activator given to a subject will depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. Dosage and dosing schedule can be determined by a medical practioner.

In some embodiments, a FXR pathway activating agent (e.g., ones described herein) may be used in combination with a second therapeutic agent (e.g., other hepatic therapeutics or anti-inflammatory agents). In some embodiments, the FXR pathway activator and the second therapeutic agent may be formulated in one pharmaceutical composition. In other embodiments, they may be formulated in separate pharmaceutical compositions. The FXR pathway activator may be administered to the subject before, after, or simultaneously with the second therapeutic agent.

### Kits for Use in Alleviating Liver Injury

The present disclosure also provides kits for use in treating or alleviating liver injury as described herein. Such kits can include one or more containers comprising an FXR pathway activator, e.g., any of those described herein. In some instances, the FXR pathway activator may be co-used with a second therapeutic agent.

In some embodiments, the kit can comprise instructions for use in accordance with any of the methods described herein. The included instructions can comprise a description of administration of the FXR pathway activator, and optionally the second therapeutic agent, to treat, delay the onset, or alleviate a target disease as those described herein. The kit may further comprise a description of selecting an individual suitable for treatment based on identifying whether that individual has the target disease, e.g., applying a routine procedure to identify the subject as suitable for the treatment. In still other embodiments, the instructions comprise a description of administering an antibody to an individual at risk of the target disease.

The instructions relating to the use of an FXR pathway activator generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating a liver injury. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an FXR pathway activator as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985>>; Transcription and Translation (B. D. Hames & S. J. Higgins, eds. (1984>>; Animal Cell Culture (R. I. Freshney, ed. (1986>>; Immobilized Cells and Enzymes (1RL Press, (1986>>; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Adaptive Transport of Bile Acids Induced by Loss of Bile Salt Export Pump Regulates Bile Acid Synthesis in Induced Hepatocytes

The goal of this study was to gain a greater understanding of the pathogenic mechanisms of genetic cholestatic liver diseases. Prominent among the subset of genetic diseases are defects in Bile Salt Export Pump (BSEP). Deficiency of this transporter is known to present in several clinical phenotypes, including Progressive Familial Intrahepatic Cholestasis type 2 (PFIC2), Benign Recurrent Intrahepatic Cholestasis type 2 (BRIC2), and Intrahepatic Cholestasis of Pregnancy (ICP). Strautnieks et al., Gastroenterology 134:1203-1214 (2008); and Strautnieks et al., Nature Genetics 20:233-238 (1998). PFIC2, the most severe form, has a wide spectrum of clinical manifestations—most commonly newborn cholestasis with varying rates of progression of the liver dysfunction. Nicolaou et al., Journal of Pathology 226:300-315 (2012). Patients with PFIC2 are also known to develop malignant transformation of hepatocytes during the first decade of life. Knisely et al., Hepatology 44:478-486 (2006). There are no therapeutic agents that have been found to be significantly effective for treatment of patients with severe PFIC2 because the specific alterations in the bile acid transport remain unclear.

To delineate the pathologic and compensatory alterations in BSEP deficient hepatocytes, several attempts have been made to generate rodent models that can recapitulate the phenotypes observed in patients with PFIC2. In the liver of the BSEP knock-out mouse, expression of ABCB1/MDR1, a transporter of bile acids at the bile canaliculus, is significantly increased, suggesting one compensatory mechanism to reduce the intracellular bile acid concentration via canalicular excretion. Wang et al., Hepatology 38:1489-1499 (2003). However, in analysis of gene expression of the human liver of patients with PFIC2, this MDR1 compensatory response was not evident. Keitel et al. Hepatology 41:1160-1172 (2005). Furthermore, since the bile of patients with PFIC2 contains a minimal amount of conjugated bile acids, BSEP deficient human hepatocytes seemingly lack the compensatory bile acid transporter on the canalicular membrane. Jansen et al., Gastroenterology 117:1370-1379 (1999).

Simple cultures of human hepatocytes fail to form functional apico-basolateral polarity, thus it has been difficult to investigate bile acid transport in human hepatocytes due to the lack of a suitable experimental system for dynamic tracing of transcellular transport of bile acids. Study of de novo bile acid synthesis by cultured hepatocytes has only been possible with a primary cell culture of explanted liver. Because an explanted liver from patients with PFIC2 is rarely available, an experimental investigation into the regulatory mechanism of bile acid synthesis and transport in human BSEP deficient hepatocytes has not been possible.

To overcome this difficulty, the present study used human induced pluripotent stem cells (iPSCs) and developed an in vitro culture system where iPSCs were differentiated into hepatocyte-like cells on a permeable membrane of a two-chamber (Transwell) system. The in vitro culture system disclosed in the Example here is an improvement of the in vitro system disclosed in Asai et al., Development 144:1056-1064 (2017), wherein inter alia, the instant in vitro culture system provides a disease model produced with a single population of cell, i.e., does not require co-culture with other cell types. Using this system, the present study investigates the fate of intracellular bile acids and their role as a mediator between de novo bile acid synthesis and transcellular transport.

Taken together, the instant study has provides an in vitro disease model for BSEP deficiency. The results reported herein provide new insights into molecular mechanisms that underlie the pathophysiology of BSEP deficiency and provide targets for therapeutic intervention in patients with PFIC2.

### Methods

### Genotype Selection and Description of the Index Case

Deleterious mutations of BSEP/ABCB11 were searched in a cohort of patients with progressive familial intrahepatic cholestasis type 2 (PFIC2). The patients in the cohort of this study had compound heterozygous mutation in BSEP, including R1090X and R928X; both are nonsense truncating mutations. One set of siblings who had an identical genotype of ABCB11; c.2782 C>T (R928X) and c.3268 C>T (R1090X) were identified. Because their parents were heterozygous for each truncating mutation, the genetic test indicates compound heterozygous mutations. Both siblings presented with severe cholestasis and required liver transplant before age of 1 year. To investigate the biological impact of a severe mutation in bile acid efflux, the R1090X truncating nonsense mutation was selected, which was reported in previous cases as a homozygous genotype. Strautnieks et al., Gastroenterology 134:1203-1214 (2008); Strautnieks et al., Nature Genetics 20:233-238 (1998); and Zhou et al., Journal of Proteome Research 14:4844-4850 (2015). Liver tissues from the subject were obtained from the explanted liver.

Cell Culture and Differentiation of iPSCs to Hepatocyte-Like Cells

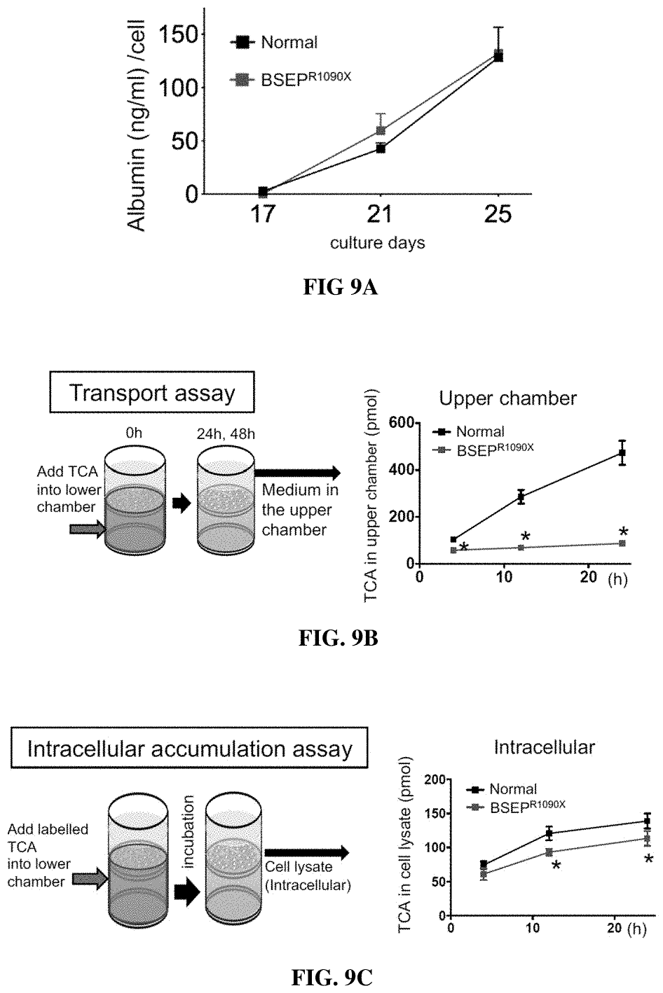

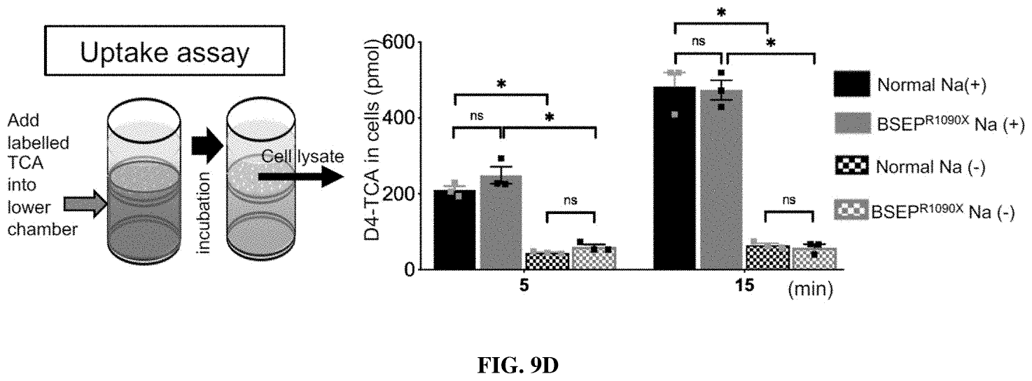

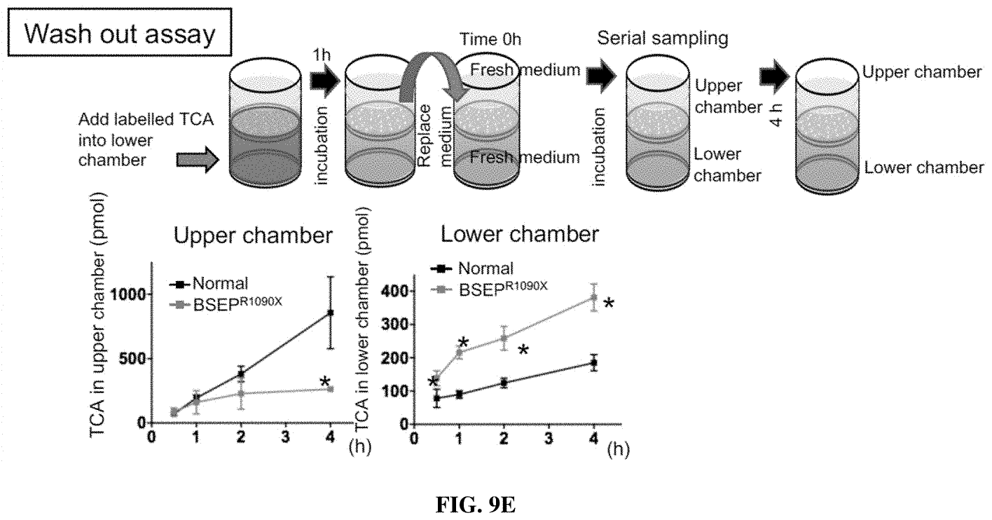

All chemical materials were purchased from Sigma-Aldrich (St. Louis, Mo.) unless otherwise indicated. All cells were incubated at 37° C. in a humidified 5% CO₂. The iPSCs (clone code: 1383D6) were derived from a healthy donor with thorough characterization of pluripotency and karyotype. Takayama et al., Hepatology Commun 1:1058-1069 (2017). For the reproduction of the study, experiments were performed on another iPSC clone (clone code=TkDA3, provided by K. Eto and H. Nakauchi). The results are summarized in FIGS. 9A-11C. One of the siblings with PFIC2 donated the peripheral blood under the IRB-approved consent. iPSCs were generated from the donated cells with a standard method of Yamanaka 4-factor transfection. Protocols for endoderm differentiation, hepatic specification, and hepatocyte maturation are modified from previously described protocols. Asai et al., Development 144:1056-1064 (2017). Briefly, for definitive endoderm differentiation, iPSCs were dissociated with Accutase and plated onto a Laminin 511 (Matrixsome, Osaka, Japan) coated cell culture dish. The medium was replaced with RPMI1640 (ThermoFisher, Waltham, Mass.) containing 2% B27 (ThermoFisher), 1 mM sodium butyrate (for the first 3 days), Wnt3a 50 ng/mL (R&D systems, Minneapolis, Minn.) and Activin 100 ng/mL (R&D) for 6 days. For hepatic specification, cells were further treated with FGF2 10 ng/mL (R&D) and BMP4 20 ng/mL (R&D) for 3 days. Cells were dissociated with TrypLE (ThermoFisher) and were plated on the membrane of Transwell insert (Corning, Corning, N.Y.). Then, cells were cultured in Hepatocyte Culture Medium (HCM) (Lonza, Allendale, N.J.) for 12 days. HCM is supplemented with HCM BulletKit (Lonza): transferrin, hydrocortisone, BSA-fatty acid free (BSA-FAF), ascorbic acid, insulin, GA-1000, and omitting human epidermal growth factor. 10 ng/mL recombinant hepatocyte growth factor (HGF), 100 nM dexamethasone, and 5% of fetal bovine serum (ThermoFisher) were added to supplement HCM.

In order to monitor the efficiency of the hepatic differentiation, the albumin production measured by ELISA assays of the culture supernatant of the hepatocyte-like cells were quantified two days prior to the experiments. HGF was removed from the medium 3 days prior to the experiments when indicated. The general scheme for producing hepatocyte-like cells from iPS cells is shown in Table 1 below.

TABLE 1
Differentiation Scheme
Endoderm differentiation medium 1 - Day 1 (On day 1, iPSCs were
dissociated with Accutase (modified trypsin) and re-plated on a
regular plastic culture dish coated with Laminin 511 or Matrigel).

| | | | | Volume |
|---|---|---|---|---|
| Reagent | Storage | Stock Conc. | Final Conc. | (per 1ml) |
| RPMI + HEPES | 4 C. | N/A | N/A | 1 ml |
| B27 (+ insulin) | −20 C. | X50 | | 20 ul |
| Activin A | −80 C. | 100 ng/ul | 100 ng/ml | 1 ul |
| Wnt3a | −80 C. | 50 ng/ul | 50 ng/ml | 1 ul |
| Y 27632 | −80 C. | 10 mM | 10 um | 1 ul |

Endoderm differentiation medium 2 - Day 2

| | | | | |
|---|---|---|---|---|
| RPMI + HEPES | 4 C. | N/A | N/A | 1 ml |
| B27 (+ insulin) | −20 C. | X50 | | 20 ul |
| Activin A | −80 C. | 100 ng/ul | 100 ng/ml | 1 ul |
| Wnt3a | −80 C. | 50 ng/ul | 50 ng/ml | 1 ul |
| Sodium Butyrate | −80 C. | 10 mM | 10 um | 1 ul |

Endoderm differentiation medium 3 - Day 3 and Day 4

| | | | | |
|---|---|---|---|---|
| RPMI + HEPES | 4 C. | N/A | N/A | 1 ml |
| B27 (+ insulin) | −20 C. | X50 | | 20 ul |
| Activin A | −80 C. | 100 ng/ul | 100 ng/ml | 1 ul |
| CHIR99021 | −80 C. | 20 mM | 3 uM | 0.15 ul |
| Sodium Butyrate | −80 C. | 500 mM | 500 uM | 1 ul |

Endoderm differentiation medium 4 - Day 5 (on this day, cells are
dissociated with trypsin and re-plated on a
permeable membrane of a Transwell)

| | | | | |
|---|---|---|---|---|
| RPMI + HEPES | 4 C. | N/A | N/A | 1 ml |
| B27 (+ insulin) | −20 C. | X50 | | 20 ul |
| Activin A | −80 C. | 100 ng/ul | 100 ng/ml | 1 ul |
| CHIR99021 | −80 C. | 20 mM | 3 uM | 0.15 ul |
| Y 27632 | −80 C. | 10 mM | 10 uM | 1 ul |

Endoderm differentiation medium 5 - Day 5

| | | | | |
|---|---|---|---|---|
| RPMI + HEPES | 4 C. | N/A | N/A | 1 ml |
| B27 (+ insulin) | −20 C. | X50 | | 20 ul |
| Activin A | −80 C. | 100 ng/ul | 100 ng/ml | 1 ul |
| CHIR99021 | −80 C. | 20 mM | 3 uM | 0.15 ul |

Hepatic specification medium - Day 7, 8, and 9

| | | | | |
|---|---|---|---|---|
| RPMI + HEPES | 4 C. | N/A | N/A | 1 ml |
| B27 (+ insulin) | −20 C. | X50 | | 20 ul |
| bFGF | −80 C. | 100 ug/ml | 50 ng/ml | 0.5 ul |
| BMP4 | −80 C. | 50 ug/ml | 20 ng/ml | 0.4 ul |

Hepatocyte maturation medium - Day 10-22

| | | | | |
|---|---|---|---|---|
| HBM Basal Media + | | | | 1 ml |
| HCM* | | | | |
| FBS | −20 C. | | 5% | 50 ul |
| Dexamethasone | −80 C. | 2.5 mM | 0.1 uM | 0.04 ul |
| HGF | −80 C. | 50 ug/ml | 20 ng/ml | 0.4 ul |

*HCM Hepatocyte Culture Media (Lonza CC-3198) and uses all components according to the bullet kit reipe but omits the EGF. FBS (Fetal bovine serum): complement heat inactivated.

CRISPR/Cas9 Genome Editing of Human iPSCs

CRISPR/Cas9 was used to introduce the truncating mutation of BSEP/ABCB11 in 1383D6 iPSCs. Candidate sgRNA target sites were selected according to the on- and off-target prediction scores from the web-based tool, CRISPOR (http://crispor.org/). The selected sgRNAs were cloned into the pX458M-HF vector that was modified from the pX458 vector (addgene #48138) and carried an optimized sgRNA scaffold and a high-fidelity Cas9 (eSpCas9 1.1)-2A-GFP expression cassette. The editing activity of the plasmid was validated in 293T cells by T7E1 assay. Kumar et al., Plos One 5 (2010); Chen et al., Cell 155:1479-1491 (2013); and Aymaker et al., Science 351:84-88 (2016). A phosphorothioated single stranded oligonucleotide-DNA (ssODN) was designed to include the intended mutations, silent mutations (to block sgRNA retargeting and to create a new restriction enzyme site for genotyping), and homologous sequence. A single cell suspension of iPSCs was prepared using Accutase and 1×10^{e6} cells were nucleofected with 2.5 μg of the plasmid and 2.5 μg of ssODN using program CA137 (Lonza). Forty-eight hours later, transfected cells were sorted one cell per well into 96 well plates based on the GFP expression. The cell clones were expanded and selected by a screening of restriction enzyme digestion. The correctly edited clones were selected based on the gain of the restriction enzyme sites on both alleles and further confirmed by Sanger sequencing for identification of bi-allelic single nucleotide mutations. Cell clones that went through the same targeting process but remained unedited were expanded and used as isogenic parental controls.

### Measurement of Bile Acid Concentration in Culture Medium

The concentration of total bile acid in culture supernatant was determined by Diazyme TBA assay (Diazyme Laboratories, Poway, Calif.) following the manufacturer's instructions. For tracer experiments, stable isotope labelled taurocholic acid (sodium taurocholic acid, [2, 2, 4, 4-²H₄]TCA, here referred to as D4-TCA)) was purchased from Cambridge isotope laboratories (Tewksbury, Mass.). For long term transport assay, D4-TCA was added into the culture medium in the lower chamber at 1 μM and 10 μM. After incubation, the supernatant of upper and lower chambers was collected. For uptake and washout experiments, cells were incubated with buffer containing 118 mM NaCl, 23.8 mM NaHCO₃, 4.83 mM KCl, 0.96 mM KH₂PO₄, 1.20 mM MgSO₄, 12.5 mM HEPES, 5 mM glucose and 1.53 mM CaCl₂). After 15 minutes of pre-incubation, D4-TCA (10 μM) containing buffer was added to the lower chambers. For uptake experiments, at 5 min and 15 min, cells were collected and frozen. For sodium-free buffer, sodium was replaced by choline (choline chloride or choline bicarbonate). For washout experiments, after 1 h of incubation with D4-TCA containing buffer, cells were washed with buffer and placed in a fresh buffer. The supernatant was then collected at 5 min, 15 min, 30 min, and 60 min from the upper and lower chamber separately. The MRP4 inhibitor, Ceefourin1, was purchased from Abcam (Cambridge, Mass.). All the samples received a fixed amount of D4-TCDCA as an internal standard and purified by protein precipitation with Acetonitrile. A calibration curve of D4-TCA was constructed using D4-TCDCA as internal standard for quantification of D4-TCA in samples. In some experiments, the endogenous bile acids and D4-TCA concentrations were measured at the University of Tokyo after confirming the compatibility of both methods.

### Measurement of D4-TCA and Endogenous Bile Acids Concentrations by Liquid Chromatography-Mass Spectrometry (LC-MS)

Cells on membrane lysed with 500 μL methanol and buffer from upper and lower chamber were subjected to LC-MS/MS analysis to quantify the concentration of D4-TCA and endogenous bile acids. 30 μL of the prepared samples were transferred to a 1 mL 96-well plate and then mixed with 120 μL of internal standard solution (100 nM D8-TCA, Santa Cruz Biotechnology, Santa Cruz, Calif.) in methanol or D5-TCA (Toronto Research Chemicals, North York, Canada) in acetonitrile. After vortex mixing, the mixtures were filtered using FastRemover for Protein (GL Sciences, Tokyo, Japan) and transferred to 96-well plate for LC-MS/MS analysis. The sample analysis was conducted on a SCIEX 5500 tandem mass spectrometer (Applied Biosystems/MDS SCIEX, Toronto, Canada) equipped with a Prominence LC system (Shimadzu, Kyoto, Japan), and operated in electrospray ionization mode. For measurement of D4-TCA concentration, samples were injected onto a CAPCELL PAK C18 MGIII column (2 mm i.d.×50 mm, Shiseido, Tokyo, Japan) and separated with the following gradient program: 10% B for 0.3 min, 10-90% B for 1.7 min, 90% B for 1.3 min, 90-10% B for 0.1 min, and 10% B for 1.9 min. The total flow rate was 0.4 mL/min, the mobile phase was 5 mM ammonium acetate in water (A) and methanol (B), and the column temperature was maintained at 40° C. For measurement of endogenous TCA concentrations, samples were injected onto a ACQUITY UPLC BEH C18 column (2.1 mm i.d.×150 mm, Waters, Milford, Mass.) and separated with the following gradient program: 20% B for 0.5 min, 20-70% B for 10 min, 70-98% B for 0.1 min, 98% B for 0.4 min, 98-20% B for 0.1 min and 20% B for 0.9 min. The total flow rate was 0.5 mL/min, the mobile phase was 0.1% formic acid in water (A) and 0.1% formic acid in acetonitrile (B), and the column temperature was maintained at 60° C. The mass spectrometer was operated in negative multiple reaction monitoring (MRM) mode. All peak integration and data processing were performed using SCIEX Analyst (Applied Biosystems/MDS SCIEX).

### Analysis of Endogenous TCA Concentrations by Liquid Chromatography-Mass Spectrometry (LC-MS)

Quantitative analysis of endogenous taurocholic acid (TCA) in the culture medium was carried out by stable-isotope dilution LC-MS with electrospray ionization in single ion recording (SIR-MS) negative ion mode using a Waters TQ-XS triple quadruple mass spectrometer interfaced with Aquity UPLC system (Milford, Mass.). Quantification of TCA was achieved by interpolation of the area ratio of each bile acid to its corresponding stable-labeled analog against a calibration curve of known concentrations of bile acids. After exchanging culture medium, cells were incubated for 48 h. The supernatants from the upper and lower chambers were collected separately. The culture supernatants and cell lysates were extracted with reverse phase solid-phase cartridge and bile acids (synthesized TCA and exogenous D4-TCA) were quantified using each standard.

### Transmission Electron Microscopy

The monolayer cells on the Transwell membrane were fixed with 2% paraformaldehyde, 2.5% glutaraldehyde in 0.1 mol/L cacodylate, pH 7.2 for 1 hour at 4° C. Specimens were then post-fixed with 1% OsO₄ for 1 hour, dehydrated in an ethanol series (25, 50, 75, 95, and 100%), and infiltrated with dilutions of ETOH/LX-112 and then embedded in LX-112 (Ladd Research Industries, Williston, Vt.) while still on the culture membrane surface. Blocks were polymerized for 3 days at 60° C. The monolayer was ultra-thin sectioned on Reichert EM UC7 ultra-microtome (Depew, N.Y.), perpendicular to the plane of the Transwell membrane and mounted on grids, which were post-stained with uranyl acetate and lead citrate. The sections were viewed using a Hitachi H7650 electron microscope (Tarrytown, N.Y.).

### Microscopic Imaging and 3D Image Reconstruction

Immunofluorescence and light microscopy imaging were performed using an Olympus microscope and DP71 camera (Olympus, Center Valley, Pa.) and Zeiss LSM710 confocal microscope (San Diego, Calif.). 3D image reconstruction of z-stack confocal images was generated using Imaris Version 7.7 software (Bitplane, Concord, Mass.).

### Protein Quantification

Unless specified, supernatant of upper and lower chamber is collected separately at 24 hours after last medium exchange. Human albumin in the collected culture supernatant was quantified with ELISA kit (Bethyl Laboratories, Montgomery, Tex.) following the manufacture instruction. For western blotting, the cells were lysed with lysis buffer (Cell Signaling Technology, Cambridge, Mass.) with proteinase and phosphatase inhibitor cocktail. Protein extracts were resolved by 4-12% SDS-PAGE and transferred to PVDF membranes. Membranes were blocked in diluted skim milk and incubated with primary antibodies at 4° C. overnight. Membranes were then washed and incubated with the secondary antibodies for 1 h at room temperature and washed again, followed by incubation of chemiluminescence reagents. Images were captured using Chemi-doc system (Bio-Rad).

### Quantitative PCR

Total RNA was extracted from cells by the RNeasy kit (Quiagen) following the manufacturer's instructions. After measuring total RNA concentration, 500 ng of RNA were subjected to reverse transcription reactions. The real-time PCR by TaqMan probe system (gene expression master mix) and the QuantStudio system (ThermoFisher) quantified mRNA of target genes, with specific primers and quantification protocol. After normalized with a housekeeping gene (18S rRNA), each gene expression level was described relative to normal i-Hep or baseline controls.

### Statistics

All in vitro experiments were performed at least in triplicate. Experimental values are expressed as mean±SEM, and statistical significance was determined by 2-tailed Student's t test or by 2-way ANOVA for comparison between 3 or more groups, followed by Bonferroni's multiple comparison post-hoc test with a significance set at p<0.05. Statistical analysis and graphic description were performed by GraphPad Prism (GraphPad Software).

### Results

(i) Generation of BSEP/ABCB11^{R1090X} Mutant Human iPSCs.

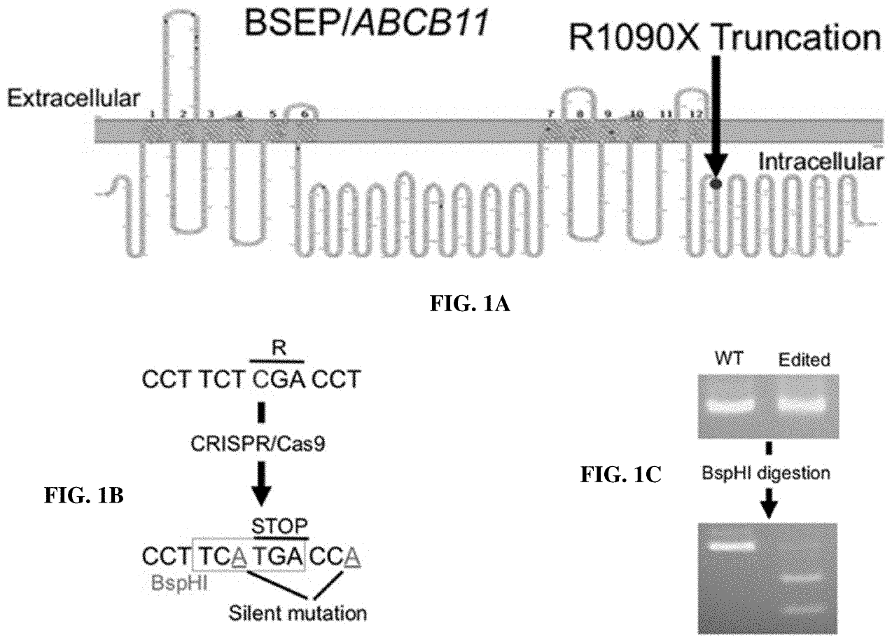

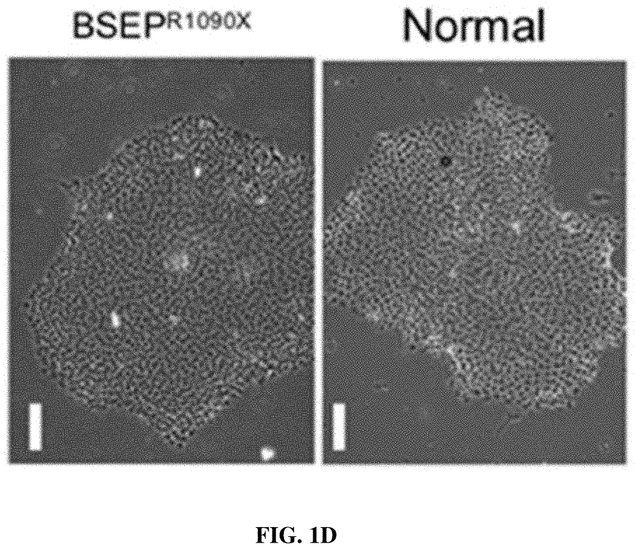

To elucidate the specific effects of R1090X truncating mutation on the BSEP function in hepatocytes, CRISPR-Cas9 genome editing was used to target the R1090 codon in the BSEP/ABCB11 gene in iPSCs obtained from a healthy donor (FIG. 1A). A single stranded oligonucleotide-DNA (ssODN) was designed to replace the codon of CGA (arginine) at position 1090 with TGA (stop codon) as well as two silent mutations to create de novo BspH1 restriction sites to facilitate colony screening (FIG. 1B). After transfection of the CRISPR-Cas9-2A-GFP plasmid and ssODN, GFP⁺ cells were FACS sorted and clones were established. Correctly targeted clones were identified by BspH1 digestion and the introduction of homozygous R1090X mutation was confirmed by Sanger sequencing (FIG. 1C). To evaluate whether the CRISPR manipulation affected their pluripotency of iPSCs, size and shape of their colonies were monitored. Parental and R1090X edited iPSCs showed comparable colony morphology (FIG. 1D). The expression of OCT4, a marker of pluripotent cells, remained comparable in parental and R1090X edited iPSCs.

To increase clinical relevance and investigate whether this system modeled BSEP deficiency in the patient's own genomic background, iPSCs from peripheral blood cells of one of the siblings were generated. This patient-origin iPSC line underwent the same evaluation for its pluripotency described above. The iPSC was labelled as iPSC^{patient}, and its BSEP genotype was BSEP^{R927X/R1090X}.

(ii) BSEP^{R1090X} iPSCs Differentiate into Hepatocyte-Like Cells and Express BSEP Protein in an Altered Pattern.

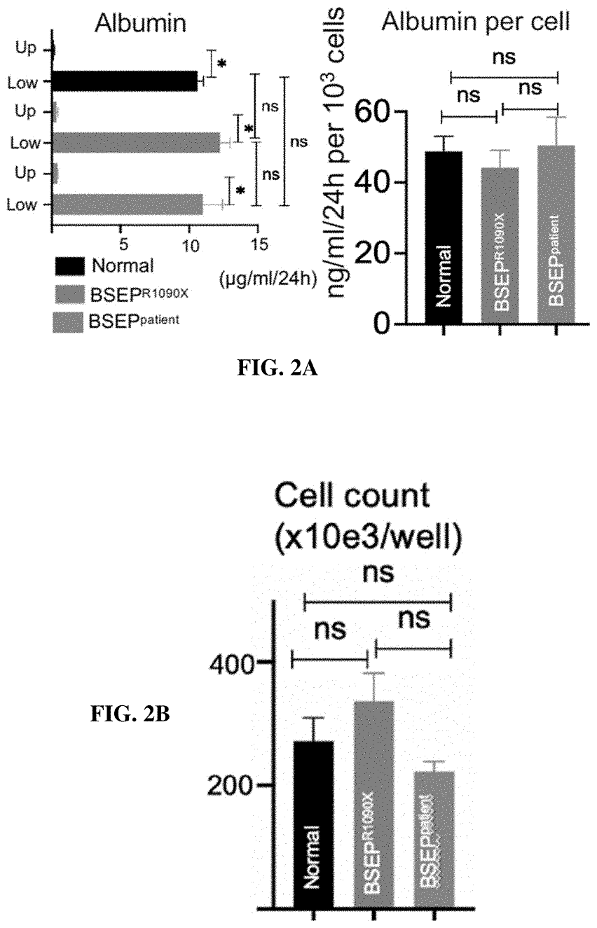

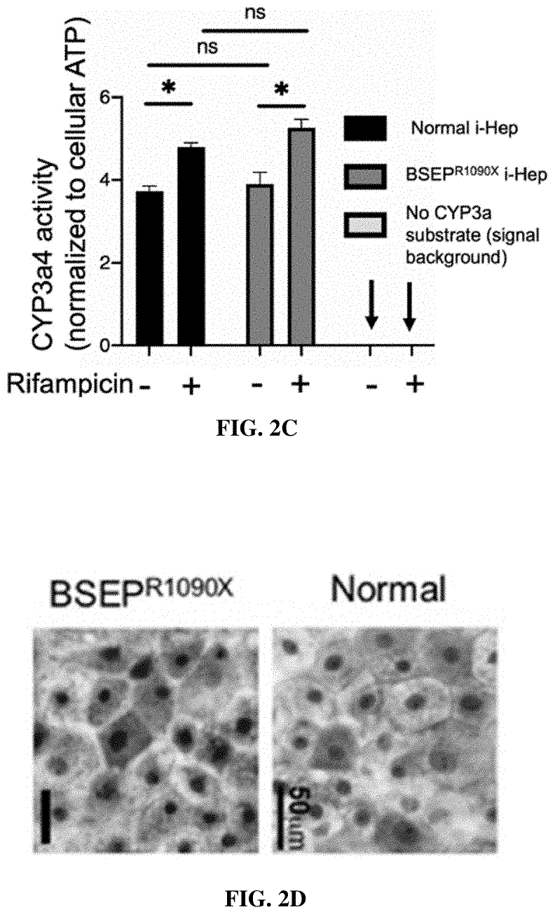

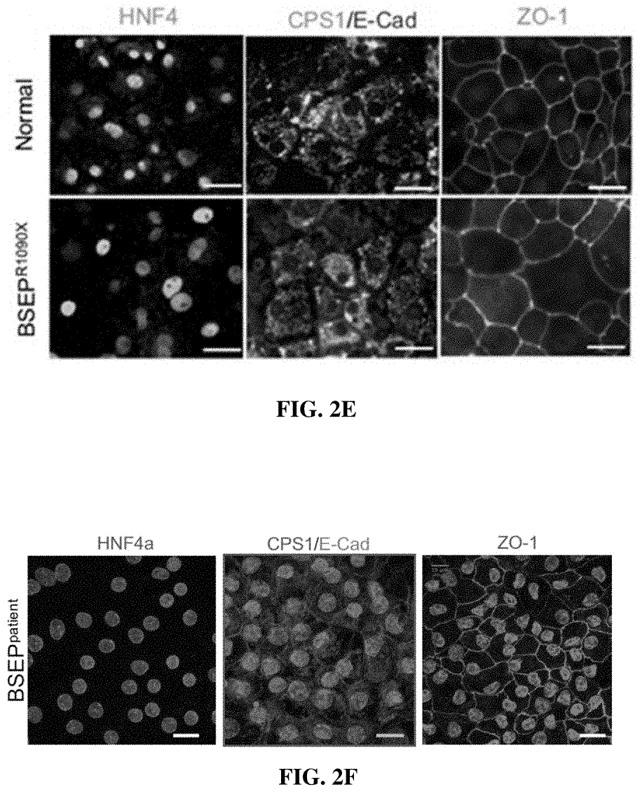

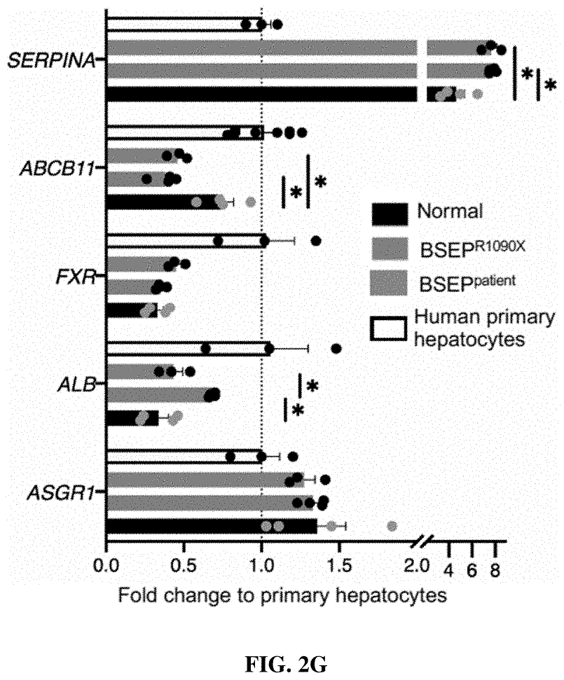

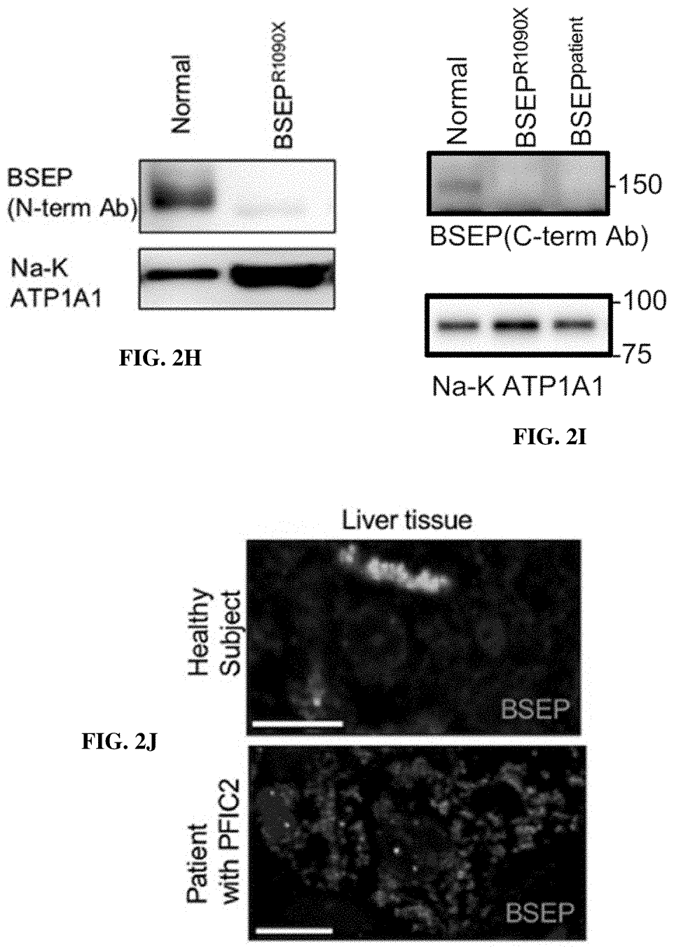

To determine whether the edited iPSCs-BSEP^{R1090X} are able to differentiate into hepatocytes, hepatic differentiation was first induced with the same method as the parental iPSCs with normal BSEP (iPSCs-BSEP normal or normal iPSCs). To quantify the efficiency of the hepatic differentiation, the albumin secretion of induced hepatocytes was measured (i-Hep). The BSEP^{R1090X} hepatocytes (BSEP^{R1090X} i-Hep) exhibited comparable albumin secretion into the culture medium to the normal i-Hep (FIG. 2A). Most of the albumin was secreted into the lower chamber (FIG. 2A, left panel). The number of cells in a well and albumin production per cell were comparable between normal and BSEP^{R1090X} i-Hep (FIG. 2A, right panel and 2B). Both i-Hep showed polygonal hepatocyte-like cells with occasional bi-nuclei formation and had comparable rifampicin-induced CYP3a4 activity (FIGS. 2C-2D). At the final stage of the differentiation protocol, BSEP^{R1090X} i-Hep expressed hepatic differentiation markers (HNF4a, CPS1) and tight junction protein (ZO1) in a pattern comparable to that of normal i-Hep (FIGS. 2E-2F).

i-Hep that were immunostained with hepatocyte marker, HNF4a. Fractions of HNF4a+ cells were determined by ImageJ cell counting tools with nuclear staining of Hoechst. These average values were statistically comparable (p>0.05). The results are provided in Table 2 below. Overall, a morphometric analysis of HNF4a+ cells showed highly efficient differentiation (>90%) in all i-Heps.

TABLE 2
Levels ofHNF4a⁺ Cells

| iHeps | HNF4a⁺ cells (%) |
|---|---|
| Normal | 93.7 ± 1.9 |
| BSEPR^{1090X} | 94.1 ± 2.1 |
| BSEPR^{patient} | 93.6 ± 2.5 |

To determine the pattern of cellular polarity, a co-immunostaining of i-Hep with F-actin was performed (relatively concentrated on the canalicular membrane of hepatocytes in the human liver tissue), Na-K transporting ATPase a1 (ATP1A1: expressed on the basolateral membrane in hepatocytes), and ZO1 (expressed between the canalicular and basolateral membrane) and analyzed their z-stack confocal images. F-actin was detected mainly on the apical membrane in both normal and BSEP^{R1090X} i-Hep, with a lower degree of expression on the lateral membrane. ATP1A1 was detected on the lateral membrane, while the basal membrane was not depicted by our confocal microscope settings due to the optical interference of the Transwell membrane. ZO1 was detected at the corner of the cells where apical and lateral membranes meet. These results indicate intact cellular polarity in both normal and BSEP^{R1090X} i-Hep.

To further compare the hepatocellular differentiation among the i-Heps, gene expression levels of hepatic markers were compared by quantitative PCR. Hepatocyte markers (FXR, ASGR1) were comparable among i-Heps. BSEP^{R1090X} and BSEP^{patient} i-Hep expressed more SERPINA1/alpha1 antitrypsin, another hepatocyte marker, compared to normal i-Hep. ALB/albumin was expressed most in BSEP^{R1090X}. The gene expression level of ABCB11/BSEP was less in BSEP^{R1090X} and BSEP^{patient} i-Hep, compared to normal i-Hep (FIG. 2G). The gene expression from primary cultured human hepatocytes were used as a reference.

Next, to determine whether genomic editing of the ABCB11 gene alters the BSEP protein expression pattern, western blotting and immunofluorescent staining of BSEP was performed. An antibody targeting the N-terminus of the protein detected BSEP in both normal and BSEP^{R1090X} i-Hep, though expression was clearly lower in BSEP^{R1090X} (FIG. 2H), while an antibody targeting the C-terminus of the protein detected BSEP in normal i-Hep, but did not detect BSEP in BSEP^{R1090X} and BSEP^{patient} i-Hep (FIG. 2I). The molecular weight of the BSEP^{R1090X} was lower than the normal BSEP, indicating a truncation of the BSEP.

i-HEP were subject to immunofluorescent staining followed by confocal and Z-stack reconstruction imaging, using the antibody targeting the N-terminus of BSEP. BSEP, F-actin and nuclei were detected. BSEP is localized on the apical membrane of normal i-Hep and in the cytosol of BSEP^{R1090X} and BSEP^{patient} i-Hep. F-actin is expressed on the apical and lateral membrane. In sum, immunostaining with the same N-terminal antibody revealed that the BSEP^{R1090X} i-Hep expressed BSEP protein in an aberrant pattern.

While normal i-Hep expressed BSEP mainly at the apical membrane of monolayer cells, BSEP was localized in the cytosol in a dot-like pattern in BSEP^{R1090X} i-Hep. To determine whether the apical membrane of BSEP^{R1090X} i-Hep maintains other transporters, i-Hep was stained with antibodies against MDR1 followed by Z-stack reconstruction imaging. F-actin was stained to localize the apical and lateral membrane. MDR1, F-actin, and nuclei were detected by confocal microscopy. MDR1 is localized on the apical membrane of normal i-Hep and BSEP^{R1090X} i-Hep. F-actin is expressed on the apical and lateral membrane.

In both normal and BSEP^{R1090X} i-Hep, MDR1 localized the apical membrane. To determine whether the pattern of BSEP expression reflects the cellular localization in liver tissue of patients with PFIC2, immunofluorescent staining of liver biopsy specimens using the same N-terminal antibody was performed (FIG. 2J). Compared to hepatocytes obtained from the liver of a healthy subject, where BSEP is localized at a canalicular membrane structure, BSEP in hepatocytes of the patients with PFIC2 was localized in the cytosol in a clustering pattern.

These results indicate that genomic editing of the ABCB11 gene in iPSCs results in the aberrant localization of BSEP in i-Hep, comparable to the pattern of BSEP localization seen in the hepatocytes in the liver of patients with PFIC2.

(iii) Cellular Ultrastructure in BSEP^{R1090X} i-Hep Recapitulates Hepatocyte Abnormalities in the Liver Tissue of the Patient with PFIC2.

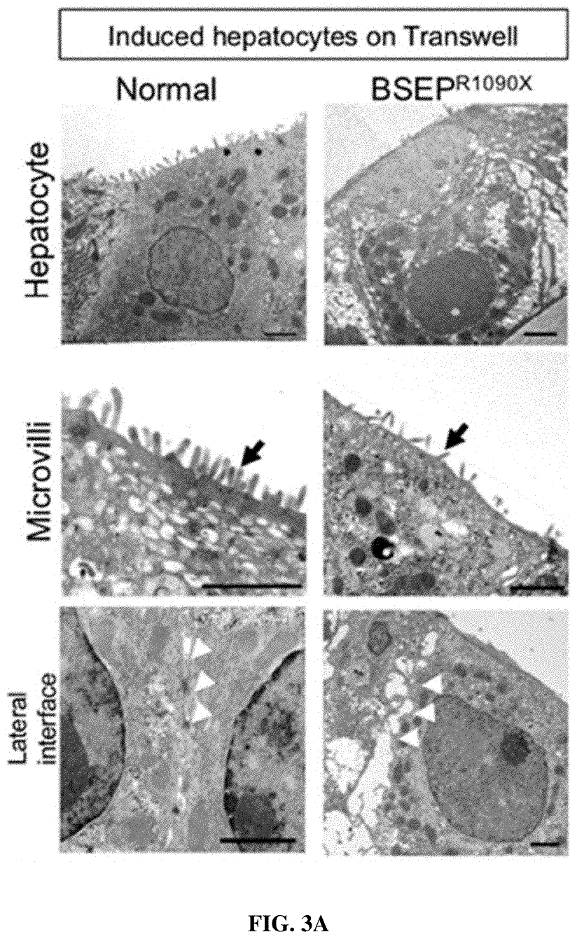

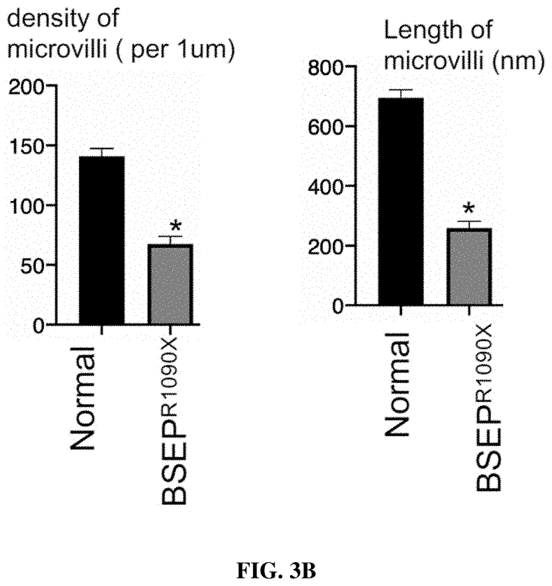

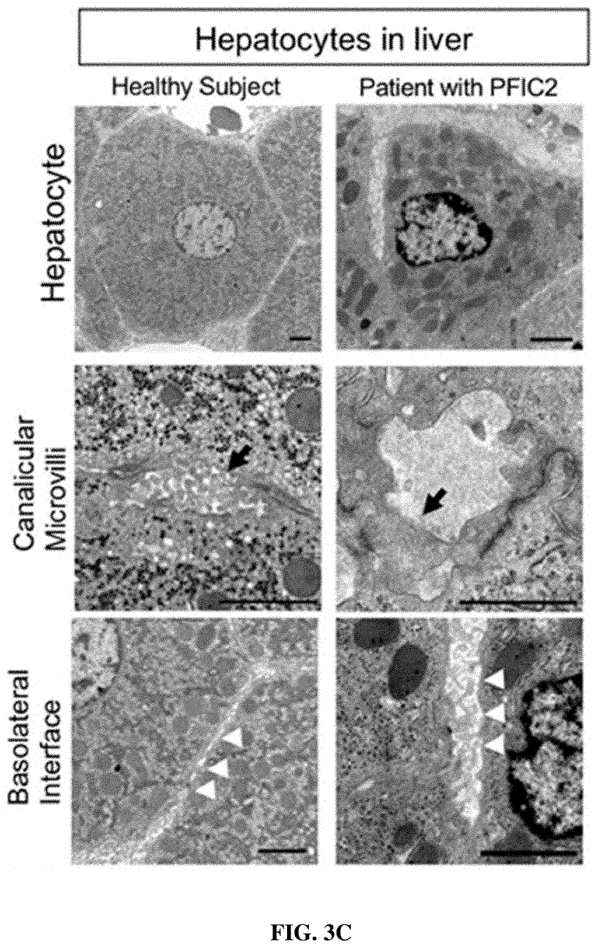

It has been reported that a development of microvilli on the bile canaliculus depends on export of bile acid across the canalicular membrane of hepatocytes. Bove et al., Pediatr Devel Pathol 7:315-334 (2004); Wolf-Pesters et al., Tissue Cell 4:379-388 (1972); and Gallin et al., Microsc Res Techniq 39:406-412 (1997). To investigate the effect of the altered BSEP expression pattern, morphological analysis of i-Hep derived from normal and BSEP^{R1090X} iPSCs was performed. To assess ultrastructural changes, i-Hep at the last stage of differentiation were evaluated by electron microscopy (FIG. 3A). Normal i-Hep showed a monolayer structure with dense microvilli on the apical membrane. These findings indicate that i-Hep developed epithelial polarization as a monolayer on the Transwell membrane, directing the apical membrane toward the upper chamber and basal interface toward the lower chamber via the permeable membrane of the Transwell. BSEP^{R1090X} showed fewer microvilli on their apical surface, indicating reduced bile acid transport across the apical membrane. FIG. 3B. Irregularity of the basolateral membrane in BSEP^{R1090X} with wider interstitial space between hepatocytes were also found. To determine whether these ultrastructural features are relevant to the patient with PFIC2 (R1090X mutation), the liver explant obtained at the time of liver transplant was investigated via electron microscopy. Compared to hepatocytes from a normal liver, hepatocytes from the patients with PFIC2 exhibited a decreased number of microvilli in the bile canaliculus and wider interstitial space between basolateral membranes of adjacent cells. FIG. 3C.

These corresponding morphological features between i-Hep and liver suggest that the pathological process of the patient with the truncating mutation manifests in BSEP^{R1090X} i-Hep.

(iv) BSEP^{R1090X} is Deficient in Exogenous Bile Acid Transport Via the Basolateral-to-Apical Phase.

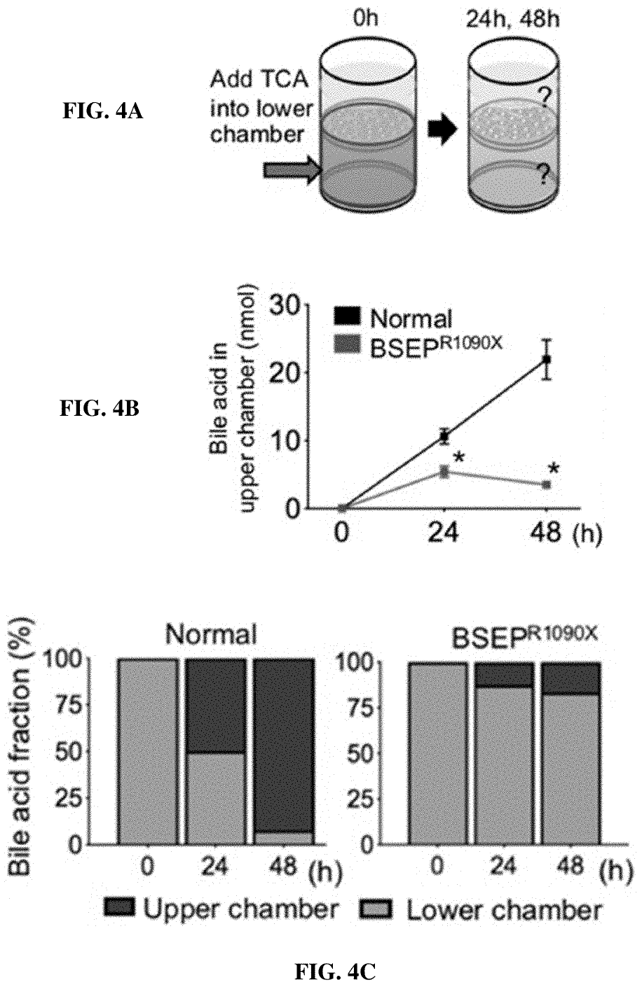

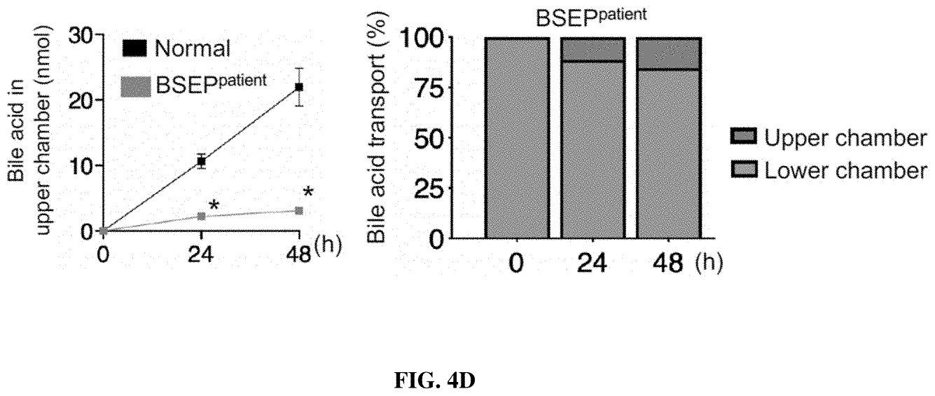

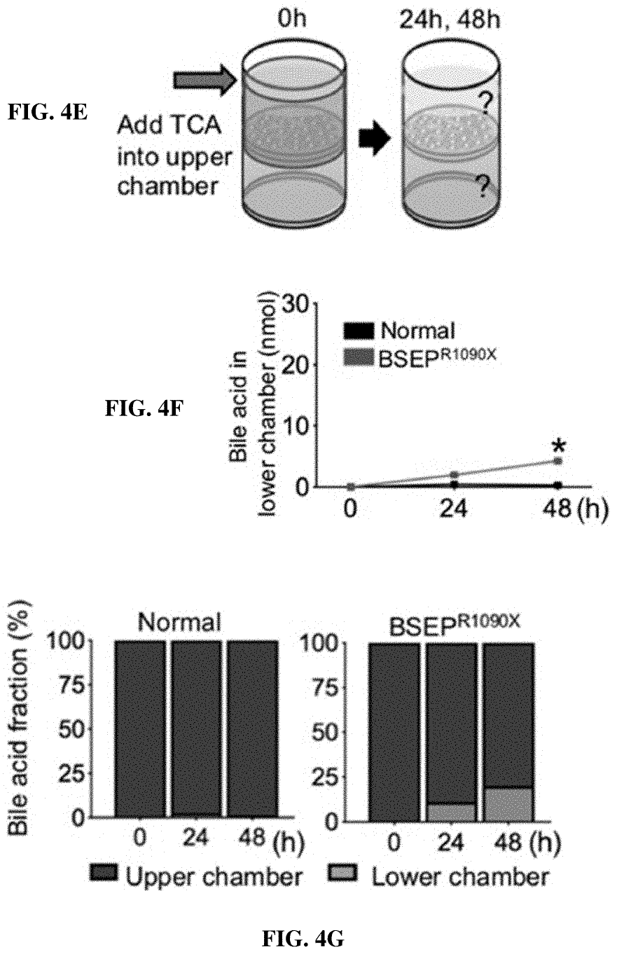

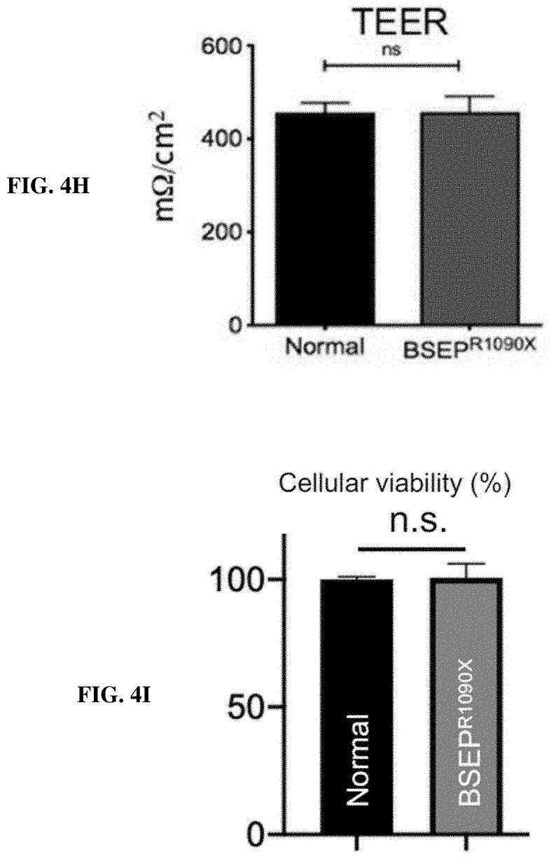

The structural defect of microvilli on the apical surface on BSEP^{R1090X} i-Hep suggested compromised canalicular function, specifically in bile acid export. To examine how the BSEP truncation impacts the exporting function of BSEP in response to exogenous bile acids, the capability of bile acid transport of i-Hep was evaluated by adding TCA to the lower chamber. In a previous study, normal i-Hep transport bile acids from the basolateral phase to apical phase was demonstrated (transcellular transport from sinusoid to bile canaliculus). To assess whether BSEP^{R1090X} i-Hep manifest altered transcellular transport of conjugated bile acids, the amount of TCA in culture medium from the upper chamber 24 hours and 48 hours after loading TCA in the lower chamber was measured. In normal i-Hep, the amount of transported TCA in the upper chamber increased at 24 h with the majority of the loaded TCA traversing from the lower to the upper chamber by 48 h (FIGS. 4A-4D). In contrast, in BSEP^{R1090X}, most of the loaded TCA remained in the lower chamber. This data formed the first indication that the transporting direction of conjugated bile acids in BSEP^{R1090X} i-Hep differs from the direction seen in normal i-Hep.

To determine whether this direction of exogenous TCA transport is specific to a basolateral-to-apical direction, the amount of TCA in the lower chamber after loading it into the upper chamber was measured (FIGS. 4E-4G). In both normal and BSEP^{R1090X}, most of the loaded TCA remained in the upper chamber. To measure the degree of paracellular “leak” of TCA, the permeability of the monolayer in normal and BSEP^{R1090X} i-Hep was compared. The following table shows the permeability of the monolayer between the upper and lower chamber measured with dextrose conjugated fluorescent probe (10,000 MW Alexa fluor). The probe was measured in the culture supernatant in the chambers 48 hours after loading into the opposite chambers; described as percentage (±SD) of the initial amount of loaded probe. Table 3 below shows the permeability data.

TABLE 3
Permeability (% of the probe leaded into the opposite chamber)

| Direction | Normal | BSEPR^{1090X} | P value |
|---|---|---|---|
| Lower to Upper | 1.2 ± 0.23% | 1.1 ± 0.19% | 0.58 |
| Upper to Lower | 0.94 ± 0.16% | 0.7 ± 0.12% | 0.42 |

After 48 hours, a minimal, comparable amount of the fluorescent probe (10,000 MW dextrose conjugated Alexa-fluoro) was transported from the lower to upper chamber in both normal and BSEP^{R1090X}. Furthermore, to compare their barrier function as a monolayer, trans-epithelial electrical resistance (TEER) between the upper and lower chamber was measured (FIG. 4H). Cellular viability was comparable between i-Heps (FIG. 4I). The resistance of the BSEP^{R1090X} monolayer was comparable to the normal i-Hep monolayer.

Together, these results demonstrate that BSEP^{R1090X} i-Hep exert a specific deficiency in basolateral-to-apical transcellular transport of conjugated bile acids.

(v) Intracellular TCA in BSEP^{R1090X} i-Hep Remains Comparable to Normal i-Hep During Transcellular Transport of TCA.

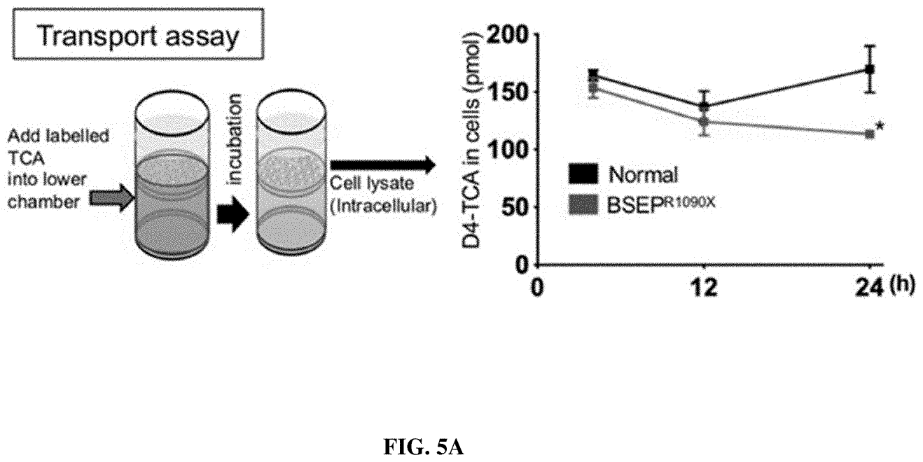

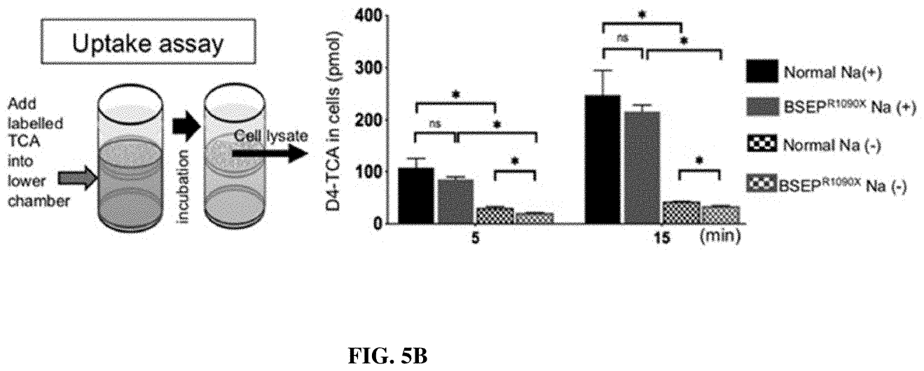

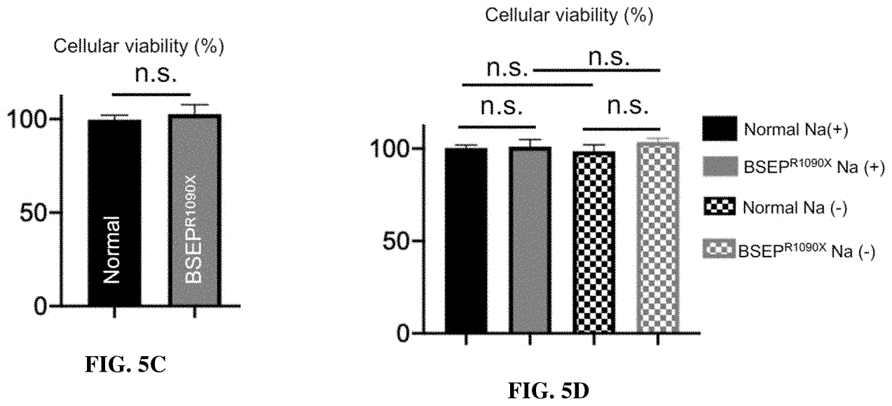

To test whether decreased bile acid export induces intracellular accumulation of TCA in BSEP^{R1090X}, molecular tracing experiments and quantified TCA concentration in cell lysates after loading TCA into the lower chamber was performed. By using isotope labelled TCA (D4-TCA), the export and uptake of TCA executed by i-Hep independent of endogenous TCA was measured. First, whether BSEP^{R1090X} i-Hep accumulate more intracellular TCA than normal i-Hep when exposed to exogenous TCA from the lower chambers was determined (FIG. 5A). To quantify the long-term transport activity, a 24-hour tracing experiment was performed. After loading D4-TCA in the lower chambers (1 μM), the amount of D4-TCA in the cell lysates was quantified at 4, 12, and 24 hours. At each time point in BSEP^{R1090X} i-Hep, the cell lysates contained comparable (4 h and 12 h) or smaller amount (24 h) of D4-TCA compared to the normal i-Hep. This result demonstrates that BSEP^{R1090X} i-Hep do not accumulate intracellular TCA to a greater degree than the normal i-Hep despite having decreased apical export of TCA.

The result prompted a test of whether BSEP^{R1090X} i-Hep have a comparable capability to uptake D4-TCA from the lower chamber by measuring intracellular D4-TCA after a short period of time before reaching their saturation. At a physiological dose of TCA (10 μM) in the culture medium in the lower chamber, normal and BSEP^{R1090X} i-Hep showed comparable amount of uptake at 5 min and 15 min of the incubation time (FIG. 5B). To test whether D4-TCA uptake was sodium dependent, intracellular D4-TCA after incubating with sodium depleted culture medium was measured. In both normal and BSEP^{R1090X} i-Hep, the intracellular D4-TCA was significantly lower compared to that in the condition of sodium containing regular culture medium. Cellular viability was comparable among conditions of i-Heps (FIGS. 5C-5D). These results indicate that BSEP^{R1090X} i-Hep exhibit comparable capability of TCA uptake in a sodium dependent fashion.

Together, the accumulation of intracellular TCA in BSEP^{R1090X} i-Hep was comparable in normal i-Hep in the setting where they uptake comparable amount of the conjugated bile acid across the basolateral surface into the intracellular space, while having deficient export of TCA across the apical membrane.

(vi) BSEP^{R1090X} i-Hep Export Intracellular TCA Via the Basolateral Membrane Toward the Lower Chamber.

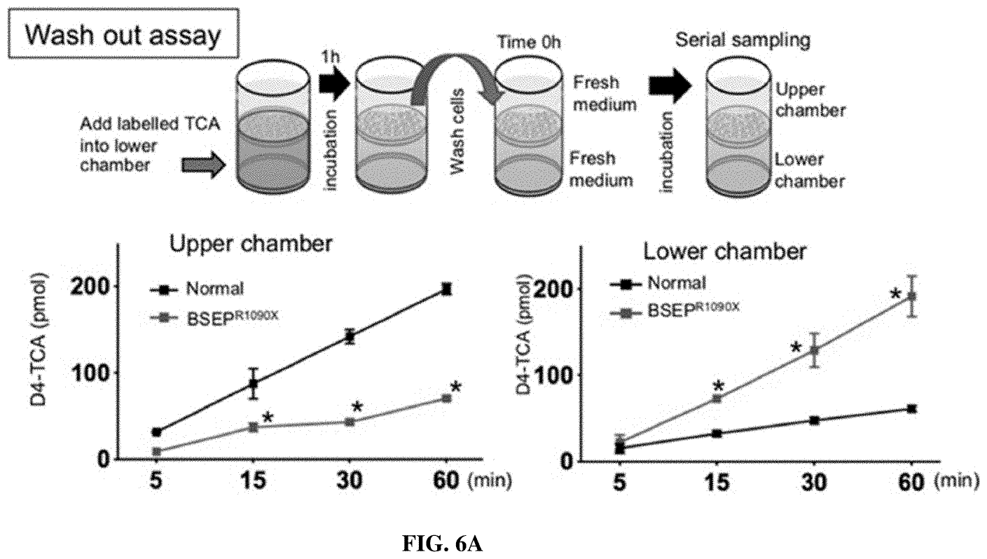

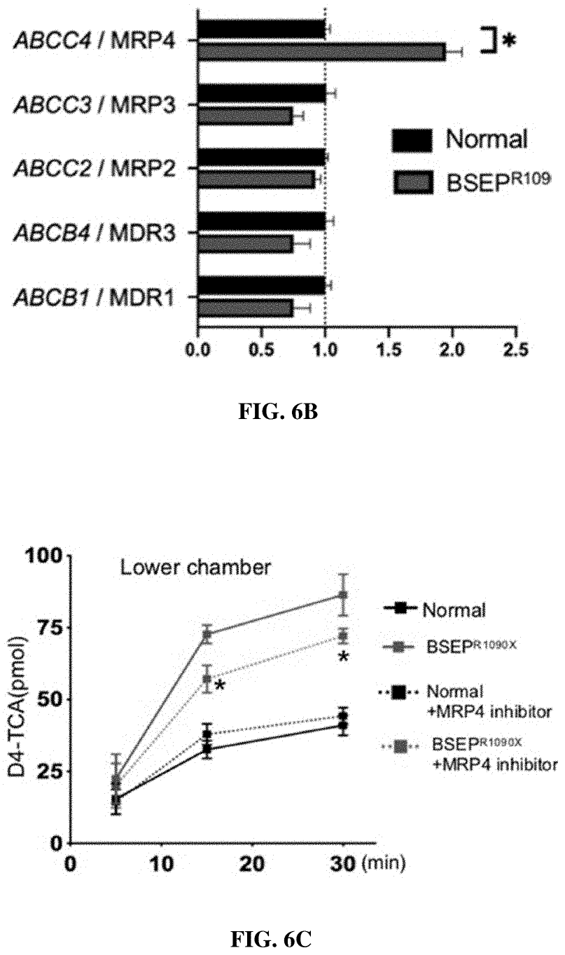

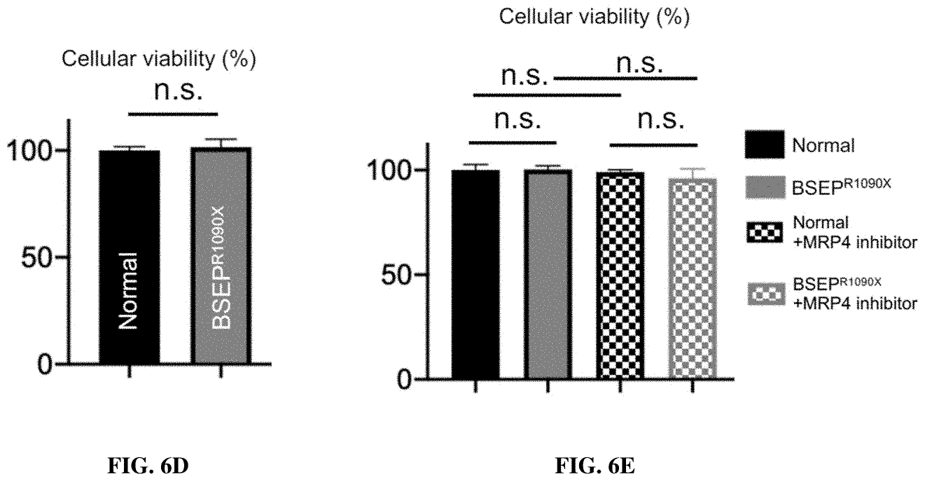

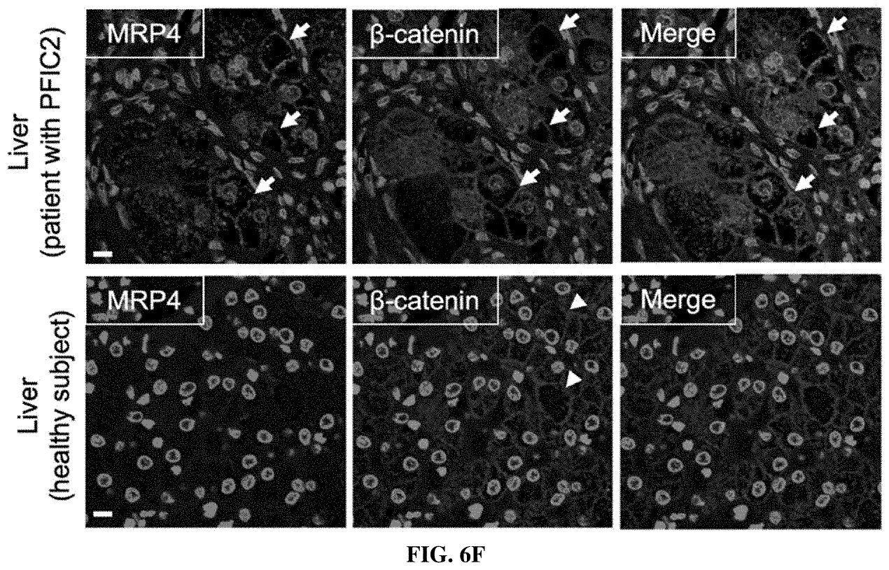

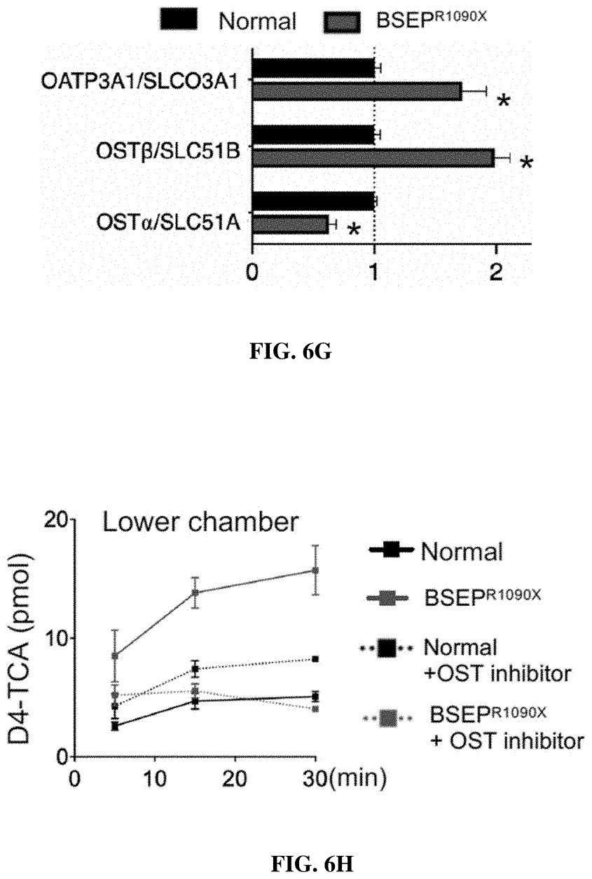

Because BSEP^{R1090X} i-Hep have a limited capacity for apical export of TCA while taking up comparable amounts of TCA, these results suggested that BSEP^{R1090X} compensates via other export channels, potentially basolateral export. To determine whether BSEP^{R1090X} i-Hep export intracellular TCA via the basolateral membrane after uptake of TCA, a “wash-out” tracing experiment with D4-TCA was performed. After one hour of incubation for uptake of D4-TCA from the lower chamber, i-Hep cells were washed gently with medium and incubated in fresh culture medium. At 5, 15, 30 and 60 minutes, D4-TCA was quantified in the upper and lower chamber to determine their export rates from the apical and basolateral membrane, respectively (FIG. 6A). The BSEP^{R1090X} i-Hep showed increased export into the lower chamber compared to normal i-Hep at each time point. In addition, BSEP^{R1090X} showed greater export toward the lower chamber than export toward the upper chamber, as seen at longer time points. The normal i-Hep showed the opposite export pattern when compared to BSEP^{R1090X} i-Hep. These results indicate that BSEP^{R1090X} i-Hep utilize basolateral export of intracellular TCA when their apical export is deficient.

To identify transporters on the basolateral membrane of BSEP^{R1090X} i-Hep, gene expressions were profiled of the transmembrane ATP Binding Cassette (ABC) transporters by quantitative RT-PCR. A gene up-regulation was found of ABCC4/MRP4—known to transport conjugated bile acids, including TCA (FIG. 6B). To further determine the functional role of MRP4 in the basolateral export of BSEP^{R1090X} i-Hep, washout tracing experiments with and without the MRP4 inhibitor (Ceefourin1) in the culture medium was performed. Cheung et al., Biochemical Pharmacology 91:97-108 (2014); and Jördens et al., Glia 63:2092-2105 (2015). Ceefourin1 decreased basolateral export of TCA in BSEP^{R1090X} i-Hep while it did not alter the basolateral export in the normal i-Hep (FIG. 6C). These results indicate that MRP4 plays a role in intracellular-to-basolateral export of TCA in BSEP deficiency. Cellular viability was comparable between i-Heps (FIGS. 6D-6E).

To determine whether hepatocytes in the patient with PFIC2 express MRP4 on the basolateral membrane, immunofluorescent staining was performed on the liver paraffin sections. MRP4/ABCC4 was detected on the plasma membrane of hepatocytes from the patient with PFIC2; this colocalized with β-catenin, indicating that MRP4 is expressed on the basolateral membrane (FIG. 6F). MRP4 was not detected on the plasma membrane of hepatocytes from the healthy subject. These results indicated that MRP4 plays a role in the intracellular-to-basolateral export of TCA in BSEP deficiency. Because MRP4 showed a partial role in the basolateral export in BSEP^{R1090X} i-Hep, the possible contribution of other transporters which are capable of carrying bile acids was investigated. Among the SLC family, OSTα/SLC51A, OSTβ/SLC51B, OATP3A1/SLCO3A1, and OATP1B3/SLCO1B3 are known to export conjugated bile acid from the basolateral domain (Alrefai and Gill, 2007; Ballatori et al., 2009; Briz et al., 2006; Bruyn et al., 2011; Pan et al., 2018). Gene up-regulation of OSTβ/SLC51B and OATP3A1/SLCO3A1 in BSEP^{R1090X} i-Hep and down-regulation of OSTα/SLC51A were found (FIG. 6G). Of note, the OSTα and OSTβ form a complex on the basolateral membrane. Both subunits are required to function; however, OSTβ plays a regulatory role for the transport function (Ballatori et al., 2013; Christian et al., 2012). The gene expression of OATP1B3/SLCO1B3 was negligible in both i-Heps. To evaluate the role of OST in the basolateral export, D4-TCA export into the lower chamber with and without OST inhibitor, clofazimine, was quantified (Malinen et al., 2018; Suga et al., 2019; Wiel et al., 2018). When OST was inhibited, the basolateral export of D4-TCA was reduced (FIG. 6H), indicating that OST plays a role in the basolateral export of TCA. It was also tested if OATP3A1/SLCO3A1 plays a role in TCA export by using the same methods; however, the role of OATP3A1 in the basolateral role in the model system was not detected (FIG. 6I). These results suggest that a combination of basolateral transporters play a role in exporting intracellular bile acids in BSEP^{R1090X} i-Hep.

Taken together, when exposed to exogenous TCA, the instant study has demonstrated that BSEP^{R1090X} i-Hep maintain low intracellular TCA concentration by export via basolateral membrane transporter(s).

(vii) Maturing BSEP^{R1090X} i-Hep Adapt an Alternative Export of Newly Synthesized Bile Acids Via the Basolateral Membrane.

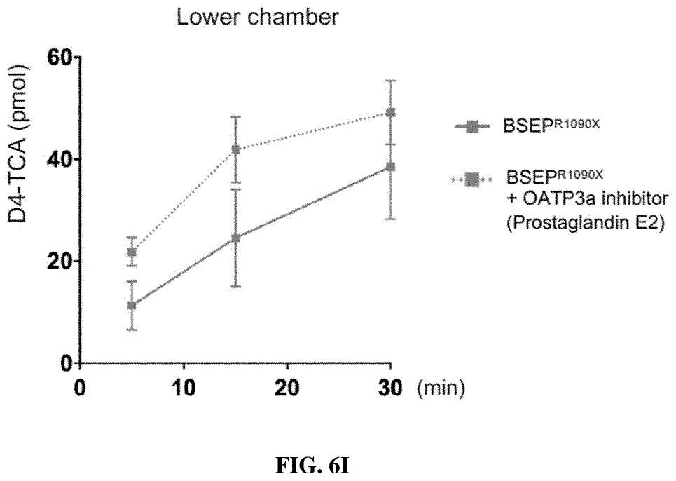

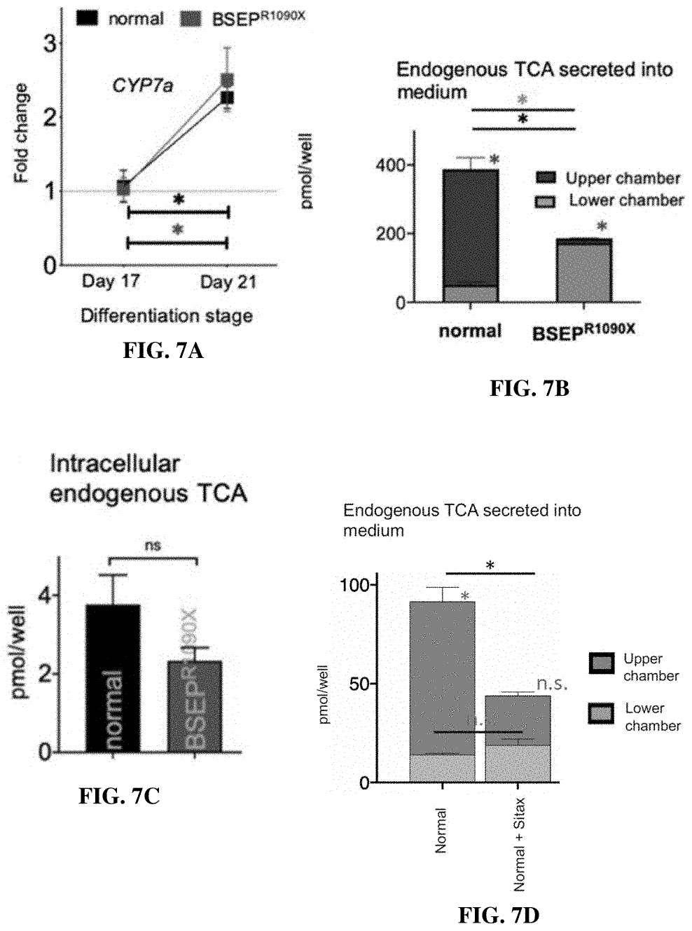

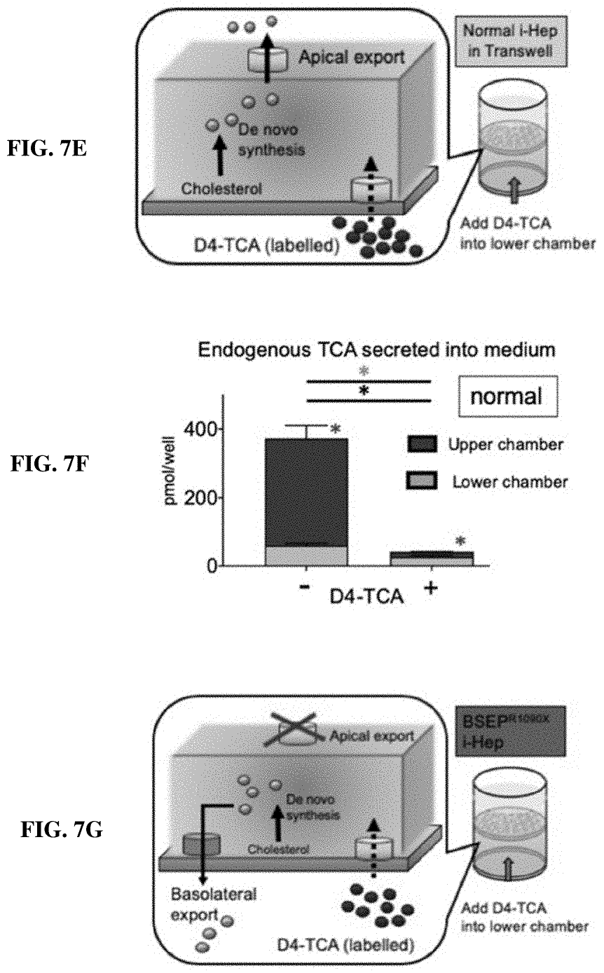

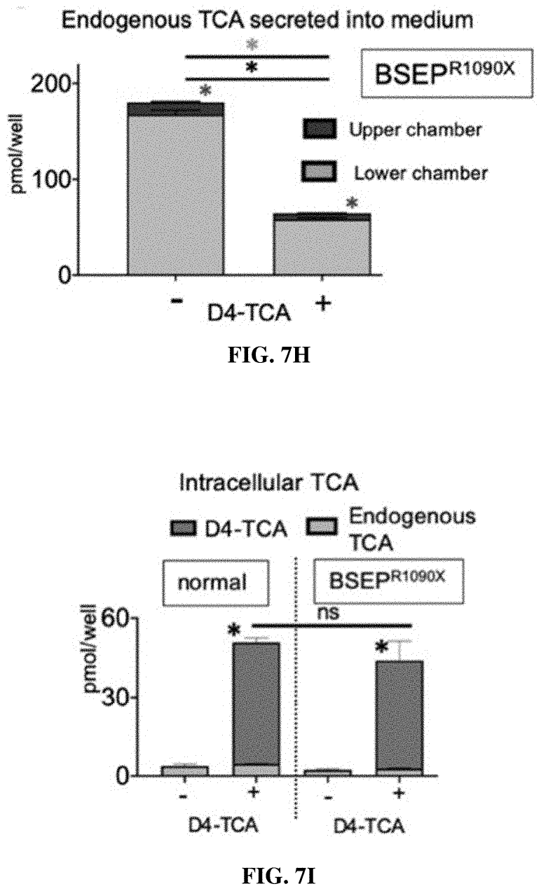

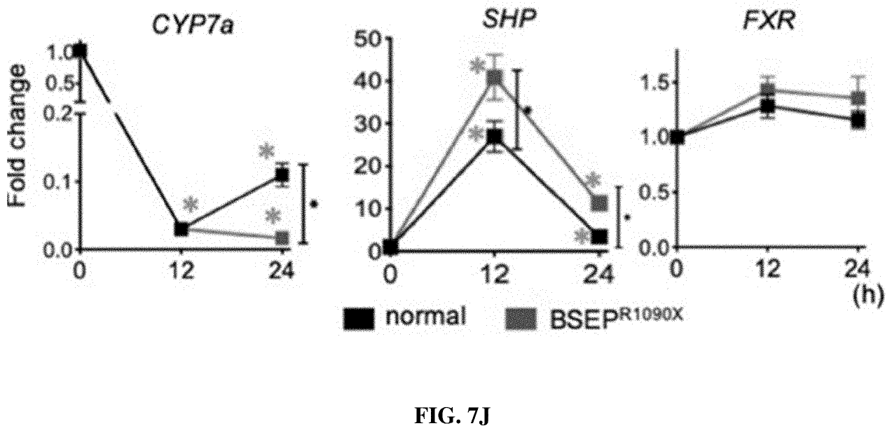

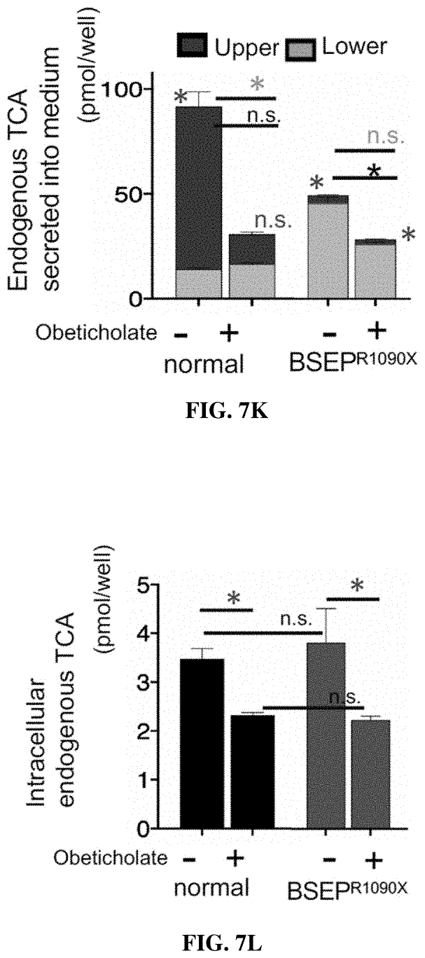

Cholestasis in patients with PFIC2 becomes prominent during the first few weeks after birth as hepatocytes initiate de novo bile acid synthesis. Based on the findings of basolateral export of exogenous bile acids, the fate of intracellular endogenous bile acids synthesized in BSEP deficient hepatocytes was investigated. To determine in which stage the i-Hep culture system induces de novo bile acid synthesis, changes in gene expression of CYP7a by RT-PCR were measured. Both normal and BSEP^{R1090X} i-Hep exhibit minimal expression of CYP7a until day 17 of the differentiation stage, then at the final stage of the differentiation (day 21) CYP7a expression is increased in both normal and BSEP^{R1090X} i-Hep (FIG. 7A). This suggests that i-Hep start synthesizing bile acids de novo at the last stage of the differentiation process.

To assess the impact of truncated BSEP on the export of intracellular bile acids synthesized de novo, the concentration of endogenous TCA secreted into the culture medium from i-Hep was measured (FIG. 7B). After 48 hours of incubation in fresh culture medium, the culture supernatant from the upper chamber and lower chamber were collected separately, as well as the cell lysates. The normal i-Hep exported more TCA into the upper chamber than into the lower chamber. This suggests that normal i-Hep predominantly export TCA via the apical membrane. Consistent with abnormal BSEP function, BSEP^{R1090X} i-Hep exported diminished amount of TCA into the upper chamber but significantly more TCA into the lower chamber, indicating that BSEP^{R1090X} i-Hep predominantly export endogenous TCA via the basolateral membrane. To further determine whether BSEP^{R1090X} i-Hep accumulate endogenous TCA in the cytoplasm, the intracellular amount of TCA in BSEP^{R1090X} and normal i-Hep was measured (FIG. 7C). BSEP^{R1090X} and normal i-Hep showed a comparable amount of intracellular TCA. To determine the specific role of BSEP in i-Hep, inhibition of BSEP in normal i-Hep was tested to see if the phenotype of BSEP^{R1090X} i-Hep was reproduced. When normal i-Hep were incubated with sitaxentan, a BSEP inhibitor (Kenna et al., 2015), the apical TCA export was reduced while the basolateral export was unchanged, resembling the trend seen in BSEP^{R1090X} i-Hep (FIG. 7D). These data indicate that maturing hepatocytes with BSEP deficiency initiate bile acid export via the basolateral membrane when de novo bile acid synthesis commences, seemingly as an adaptive mechanism to prevent the accumulation of intracellular bile acids.

(viii) Basolateral Transport of Exogenous Bile Acids Suppresses the De Novo Synthesis of Endogenous Bile Acids Via FXR Pathway in BSEP^{R1090X} i-Hep

During trans-hepatocellular transport of the sinusoidal bile acids to the bile canaliculus, de novo bile acid synthesis is suppressed. To determine whether sinusoidal bile acids in the basolateral domain suppress bile acid synthesis in BSEP deficient hepatocytes, de novo bile acid synthesis and transcellular bile acid transport using D4-TCA as an exogenous bile acid were simultaneously quantified (FIGS. 7E and 7G). The exogenous D4-TCA (10 μM) was added in the lower chamber media and was quantified by mass spectrometry, separately from the endogenous TCA. In normal i-Hep, while D4-TCA in the lower chamber was transported to the upper (data not shown), in the same time period, de novo synthesis of TCA by the normal i-Hep was significantly suppressed (FIG. 7F). In contrast, D4-TCA was minimally transported to the upper chamber in BSEP^{R1090X} i-Hep, but de novo synthesis of TCA was still significantly suppressed (FIG. 7H). To determine change of the intracellular TCA accumulation by exogenous D4-TCA, endogenous TCA and D4-TCA in the cell lysates after the incubation was measured (FIG. 7I). Normal and BSEP^{R1090X} i-Hep accumulated comparable amount of D4-TCA intracellularly. This result suggests that intracellular TCA, taken up by either normal or BSEP^{R1090X} i-Hep, regulates the rate-limiting step of bile acid synthesis.

To determine whether the regulatory effect was mediated by the FXR pathway, gene expression of FXR and its target genes were quantified, SHP and CYP7a, in i-Hep after exogenous TCA was added in the lower chambers (FIG. 7J). Both normal and BSEP^{R1090X} i-Hep exhibit FXR pathway activation, shown as an increased expression of SHP and decreased expression of CYP7a, when importing TCA. Thus, these results indicate BSEP deficient hepatocytes are able to suppress de novo bile acid synthesis via FXR pathway, when they are not transporting bile acids to the bile canaliculus.

Recently, an FXR agonist, obeticholic acid, was approved by the FDA for the treatment of cholestatic liver diseases (Jones, 2016). To determine the effect of FXR agonist on de novo bile acid synthesis of BSEP deficient human hepatocytes, the endogenous TCA production of normal and BSEP^{R1090X} i-Hep when incubated with obeticholic acid was quantified. Similar to the exogenous TCA, obeticholic acid suppressed de novo synthesis of TCA in BSEP^{R1090X} i-Hep while reducing the intracellular accumulation of TCA (FIGS. 7K-7L). This finding indicates that the BSEP deficient model system is feasible to investigate cellular mechanisms of bile acid transport and synthesis in human hepatocytes.

(ix) Similar Results were Obtained from Another iPSC Clone

Another iPSC clone (clone code: TkDA3) was generated from one of the siblings with PFIC2 following a standard method of Yamanaka 4-factor transfection. Protocols for endoderm differentiation, hepatic specification, and hepatocyte maturation are modified from previously described protocols (Asai et al., 2017). CRISPR/Cas9 genome editing was performed, as described in the main report. Sanger sequencing of the cloned iPSCs confirmed homozygous alteration of the genome sequence. As shown in FIG. 9A, hepatic differentiation of the BSEP^{R1090X}-(TkDA3)iPSC was comparable to normal (TkDA3)iPSC. Albumin secretion per i-Hep cell during the last 8 days of hepatic differentiation was compared. Normal and BSEP^{R1090X} i-Hep exhibited comparable albumin secretion into the culture medium. Immunostaining using an antibody targeting the N-terminus of BSEP reveals that BSEP is localized on the apical membrane. E-cadherin is found to be expressed on the lateral membrane.

The same TCA transportation assay as disclosed above were performed. The amount of bile acid in the medium of the upper chamber was measured by mass spectrometry at 4 h, 12 h, and 24 h after loading isotope labelled TCA (D4-TCA) in the lower chamber. (*p<0.05, n=5). BSEP^{R1090X} i-Hep showed minimal transport of D4-TCA compared to normal i-Hep up to 24 hours. BSEP^{R1090X} i-Hep also showed less intracellular D4-TCA compared to normal i-Hep at 12 and 24 hours. Further, BSEP^{R1090X} i-Hep showed comparable uptake of D4-TCA to normal i-Hep. Without sodium in the culture medium (checker boxes), D4-TCA was not taken up by the i-Hep. (*: p<0.05, n=3). The results are shown in FIGS. 9B and 9E.

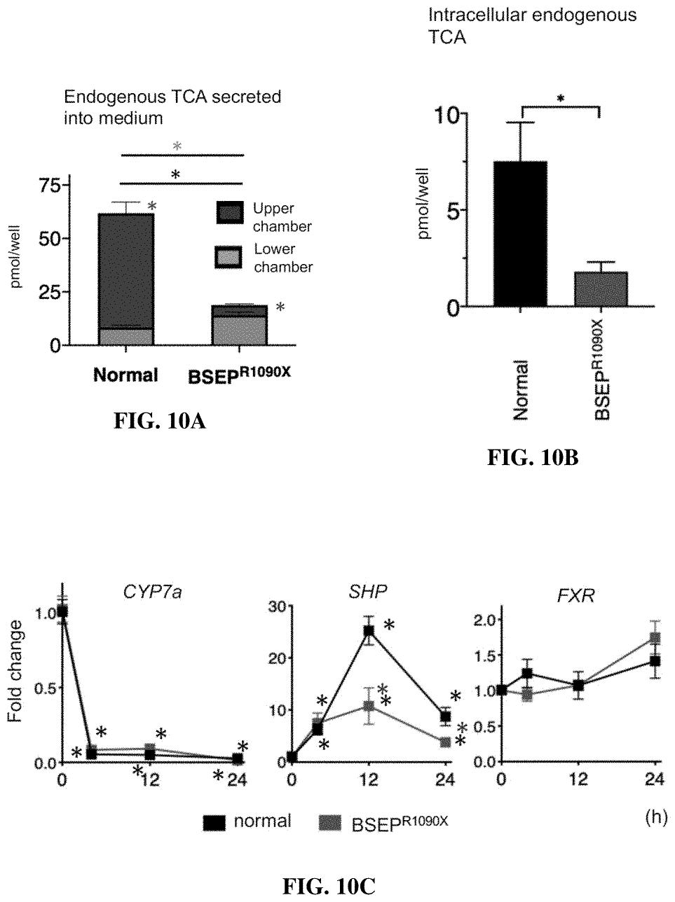

De novo bile acid synthesis was also analyzed following the same assay as disclosed above. Similar results were observed as shown in FIGS. 10A-10C. Normal i-Hep (TkDA3) exported endogenous TCA towards the upper chamber (apical domain) whereas BSEP^{R1090X} i-Hep (TkDA3) towards the lower chamber (basolateral domain). The total amount of TCA synthesized by BSEP^{R1090X} i-Hep was less than normal i-Hep (*=p<0.05, n=3 or more). Similar to the results reported above, in both normal and BSEP^{R1090X} i-Hep (TkDA3), CYP7a was down-regulated and SHP was up-regulated when TCA was added into the lower chamber for 4 h, 12 h, and 24 h. No significant change was found in FXR expression. The fold change of gene expression was based to the values in the condition cultured without TCA. (black*p<0.05, n=3 or more). When compared to normal, BSEP^{R1090X} i-Hep showed a less fold increase of the SHP gene at 12 h and 24 h. (*p<0.05).

### CONCLUSIONS

Intracellular accumulation of conjugated bile acids in BSEP deficient hepatocytes has been proposed since conjugated bile acids are not excreted in the bile and are found in the liver in high concentration. However, direct evidence of intracellular accumulation of bile acids in human hepatocytes is lacking. In this report, new insights into the mechanism of cellular regulation of intracellular bile acids are provided. By using a newly established in vitro system of human hepatocytes, which recapitulates the expression pattern of truncated BSEP, it was found that hepatocytes with BSEP deficiency in part use basolateral transporters, MRP4, to export conjugated bile acids in order to prevent their intracellular accumulation.

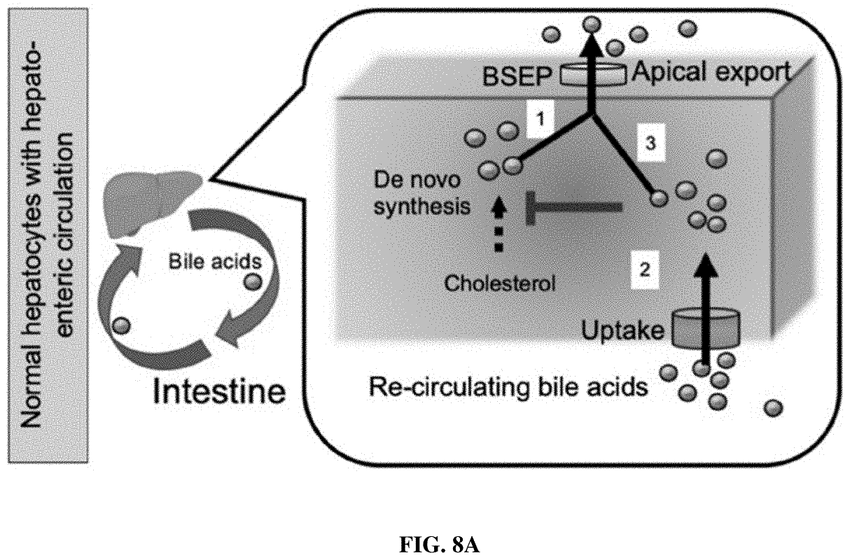

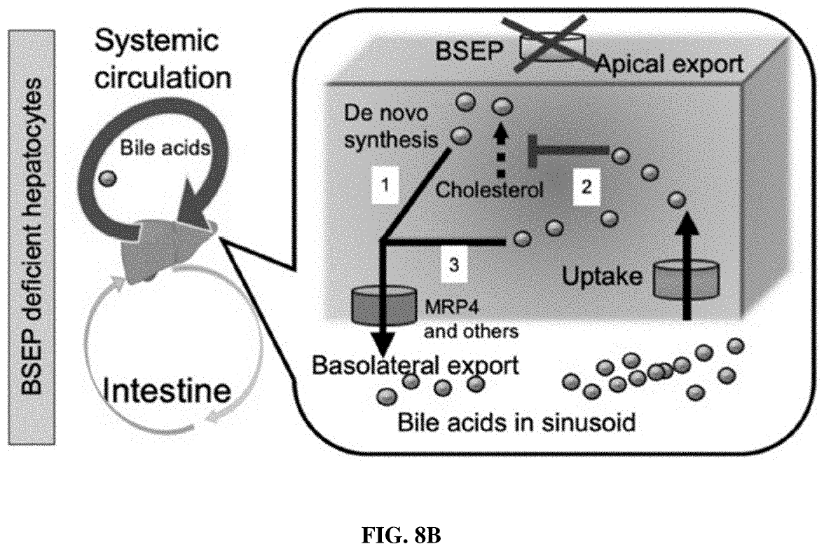

Hepato-enteric bile acid circulation reaches homeostasis by the interaction between transcellular bile acid transport and de novo synthesis mediated by intracellular bile acids in hepatocytes (FIG. 8A). i-Hep in culture system described herein synthesized de novo bile acids at the last stage of the hepatic differentiation under the regulation of HGF, consistent with previous reports of spontaneous bile acid synthesis and secretion by cultured hepatocytes. Ellis et al., Methods Mol Biology Clifton N J 640:417-430 (2010); Liu et al., Toxicol Sci 141:538-546 (2014); and Einarsson et al., World J Gastroentero 6:522-525 (2000). The present study demonstrated that human hepatocytes develop regulatory mechanisms to control the concentration of intracellular conjugated bile acids when BSEP is genomically deficient. The BSEP deficient hepatocytes export endogenous conjugated bile acids via the basolateral membrane as they mature. In patients with PFIC2, since sinusoidal bile acids do not flow into the hepato-enteric circulation, they remain in the systemic circulation, leading to jaundice and cholestasis (FIG. 8B). The mechanisms regulating bile acids accumulating in the systemic circulation and de novo bile acid synthesis have not been defined previously.

In this report, it was demonstrated BSEP deficient hepatocytes are able to down-regulate de novo bile acid synthesis via the uptake and export of bile acids on the basolateral domain, while preventing accumulation of intracellular bile acids. This suggests that BSEP deficient hepatocytes can achieve homeostasis of bile acids concentration of the systemic circulation. These findings provide potential treatment options to reduce liver injury in patients with PFIC2 by suppressing de novo bile acid synthesis with an FXR activator in the early stage of life.

The analysis of ultrastructure showed structural disturbance of the basolateral membrane in BSEP^{R1090X} i-Hep. Previous studies showed that increased concentration of bile acids increases lipid fluidity of plasma membrane and disrupt membrane functional domain. Scharschmidt et al., Hepatology 1:137-145(1981). It was speculated that constant intracellular-to-basolateral reflux of bile acid may cause abnormally increased concentration of bile acids between the lateral membranes of adjunct cells, thus induce membrane degradation or instability. Given that these changes were found in the liver of patients with PFIC2, they may be important pathophysiological features of BSEP deficiency.

This report provides a proof of concept for a novel in vitro disease model for BSEP deficiency. By generating isogenic iPSCs through CRISPR/Cas9 technology, it was able to elucidate a direct molecular consequence of a single nucleotide variant found in patients. This system allows for directly determination of the cellular and biochemical effect of previously unreported genetic variants and the molecular consequence of missense mutations, often reported as “variant of unknown clinical significance”. As the knowledge of disease-causing variants further accumulates, it would be relied on to predict the clinical course from the genotype and design personalized management strategies at an early stage of the disease.

In summary, these findings reveal novel mechanisms that underlie the pathophysiology of BSEP deficiency and provide targets for therapeutic intervention in patients with PFIC2.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

It should also be understood that, unless clearly indicated to the contrary, in any methods claimed herein that include more than one step or act, the order of the steps or acts of the method is not necessarily limited to the order in which the steps or acts of the method are recited.

1. A method for alleviating liver injury, comprising administering to a subject in need thereof an effective amount of a Farnesoid X receptor (FXR) activator.

2. The method of claim 1, wherein the FXR activiator is selected from the group consisting of ethanolamine, phosphoethanolamine, phosphatidylethanolamine, obeticholic acid (OCA), a 6α-ethyl derivative of the natural human BA chenodeoxycholic acid (CDCA), Chenodeoxycholic acid (CDCA), Fexaramine, and GW 4064.

3. The method of claim 1, wherein the subject is a human patient having a cholestatic liver disease.

4. The method of claim 3, wherein the cholestatic liver disease is benign recurrent intrahepatic cholestasis type 2, intrahepatic cholestasis of pregnancy, or progressive familial intrahepatic cholestasis type 2.

5. The method of claim 1, wherein the subject is a pediatric patient.

6. The method of claim 1, wherein the subject is a human adult patient.

7. The method of claim 1, wherein the FXR activator is administered to the subject via oral administration or injection.
